(19) United States
(12) Patent Application Publication
(10) Pub. No.: US 2015/0216779 A1
(43) Pub. Date: Aug. 6, 2015

(54) COMPOSITIONS FOR TARGETED ANTI-AGING THERAPY

(71) Applicant: THE TEXAS A&M UNIVERSITY SYSTEM, COLLEGE STATION, TX (US)

(72) Inventor: Adela Mora-Gutierrez, Houston, TX (US)

(21) Appl. No.: 14/421,520

(22) PCT Filed: Aug. 14, 2013
(86) PCT No.: PCT/US2013/054883

§ 371 Date: Feb. 13, 2015

Related U.S. Application Data
(60) Provisional application No. 61/682,988, filed on Aug. 14, 2012.

(51) Int. Cl. A61K 8/64; A61K 8/34; A61Q 19/08; A61K 8/44; A61K 8/37; A61K 8/11; A61K 8/35; A61K 8/58

(52) U.S. Cl. CPC A61K 8/64; A61K 8/355; A61K 8/34; A61K 8/58; A61K 8/44; A61K 8/37; A61K 8/11; A61K 8/645; A61Q 19/08; A61K 2800/56

(57) ABSTRACT

This invention relates to compositions for delaying aging. The compositions comprise branched-chain amino acids and whey protein, and combinations thereof, containing the same, such as L-leucine, L-isoleucine, L-valine, lactoferrin, and β-lactoglobulin. The compositions are heat stable when dissolved in water at near neutral pH. The compositions are palatable and suitable for delivering orally administrable anti-aging agents such as ω-3 fatty acids, coenzyme Q₁₀, xanthophylls, L-arginine, and L-glutathione.

### CROSS-REFERENCE TO RELATED APPLICATION

This application claims the benefit of U.S. Provisional Application Ser. No. 61/682,988, filed Aug. 14, 2012, the disclosure of which is hereby incorporated by reference in its entirety.

### FIELD OF INVENTION

The present invention concerns compositions for delaying aging.

### BACKGROUND OF THE INVENTION

Oxidative damage has long been assumed to be a major factor in mammalian aging (Harman, D. in J. Geront. 11, 298-300, 1956). Age-related muscle wasting, muscle weakness, and reduced aerobic capacity result in many metabolic disorders and diminished physical performance in humans (Rooyackers et al. in Proc. Natl. Acad. Sci. USA 93, 15364-15369, 1996; Balagopal et al. in Am. J. Physiol. 273, E790-E800, 1997; Short, K. R. and Nair, K. S. in J. Endocrinol. Invest. 22, 95-105, 1999). Reduced mitochondrial function could contribute to age-related muscle dysfunction and reduced aerobic capacity. In the most comprehensive human study yet performed, Short et al. in PNAS 102, 5618-5623 (2005) found that the content of several mitochondrial proteins was reduced in older muscles, whereas the level of oxidative DNA lesion, 8-oxo-deoxy-guanosine, was increased, supporting the oxidative damage theory of aging.

Oxidation is a normal bodily process in which heat and free energy are released for maintaining body temperature, constructing and repairing cellular structures, degrading and eliminating unwanted ones, and other metabolic processes. However, undesirable oxidation often occurs, causing damage to cells and tissues. Many conditions can exacerbate oxidative damage, including:

(a) Excessive exposure to xenobiotics,

(b) Presence of improperly sequestered and/or excessive amounts of semiquinones and transitional metals, which can cause one-electron reduction of molecular oxygen to form superoxide radicals,

(c) Photo-induced lysis of chemical bonds (or electron redistribution) to form various free radicals, which eventually lead to, in the presence of molecular oxygen, the formation of oxygen centered radicals and peroxidation,

(d) Photosensitized formation, in the presence of chromophores, of singlet oxygen, which attacks unsaturated centers of an organic molecule and abstracts a hydrogen atom, leading to the formation of hydroperoxides and peroxidation,

(e) Infections.

Even in the absence of these abnormal conditions, erroneous oxidation that damages cellular constituents may occur in the normal oxidation pathways, as dictated by the uncertainty principle of quantum mechanics. Thus the body has evolved to have arrays of anti-oxidative protective and repair mechanisms. The body's arrays of anti-oxidative devices include substances that can quench singlet oxygen and free radicals (superoxide ion, hydroxy radical, and other radicals), inactive hydrogen peroxide and hydroperoxides, and sequester transitional metal ions. They range from small antioxidant molecules, such as coenzyme Q₁₀ (CoQ₁₀) and glutathione (GSH), to complex antioxidant enzymes, such as superoxide dismutase, glutathione peroxidase, peroxidases, and catalases.

There are three potential dietary strategies for affecting the rate of aging in humans: (1) prevention and/or repair of oxidative damage, (2) reduction of caloric intake, and (3) intake of omega (ω)-3 fatty acids. To combat the destructive free radicals that are generated when we engage in such everyday activities as eating, breathing, exercising, battling a cold or disease, or exposed to pollution, cigarette smoke, or the sun's ultraviolet lights, antioxidants should be consumed with every meal. Fruits and vegetables contain powerful antioxidants. Carotenoids (xanthophylls) are among the most abundant natural antioxidant compounds, and have been reported to possess vast potential as anti-aging compounds. Among xanthophylls, lutein has recently received attention for altering thought processes that facilitate exercise. A novel nutritional strategy to prompt an increase in physical activity in older people and make exercise more enjoyable would include the intake of milk combined with lutein, which is found in green leafy vegetables. While many people associate whey protein (β-lactoglobulin is 55% of total whey protein; lactoferrin is 5% of total whey protein) with body building, it could prove a key weapon in the fight against sarcopenia. Sarcopenia is a condition that affects the older generation, and is linked to a loss of lean body mass, strength, and function. There are many health benefits associated with whey protein (Hoppe et al. in J. Nutr. 138, 145S-161S, 2008) and the positive physiological effects of the branched-chain amino acids L-leucine, L-isoleucine and L-valine (D'Antona et al. in Cell Metabolism 12, 362-372, 2010). Whey protein contains the highest concentration of branched-chain amino acids available from any food protein source.

Moreover, the restriction of caloric intake, while maintaining an appropriate intake of essential nutrients, is the most universal intervention known for the extension of life span in animals. Such diets are hard to find in real life situations. An important consideration for older individuals is that a lowered immune response is evident in populations subject to chronic low energy intakes (Charlton, K. E. in Asia Pacific J. Clin. Nutr. 11, S607-S617, 2002). The importance of senescense of the immune system is evidenced by the high incidence of tumors and the greater susceptibility to infections from pathogens shown by the aged. Thus, the immune system has been proposed as a marker of biological age and life span since a suboptimal immune system significantly contribute to morbidity and mortality in the elderly (De la Fuente, M. in Eur. J. Clin. Nutr. 56, 55-58, 2002).

With respect to the intake of ω-3 fatty acids, marine-derived ω-3 docohexaenoic acid (DHA) and eicosapentaenoic acid (EPA) supplementation influences immune function in healthy adults and patients (Trebble et al. in Br. J. Nutr. 90, 405-412, 2003), whereas others have not found any effects (Kew et al. in Am. J. Clin. Nutr. 77, 1287-1295, 2003). However, DHA/EPA supplementation has been shown to modulate immune function in healthy infants (Damsgaard et al. in J. Nutr. 137, 1031-1036, 2007). The intake of DHA/EPA may also counter degenerative muscle loss (Smith et al. in Am. J. Clin. Nutr. 93, 402-412, 2011). Healthy people in their 70s can lose as much as 10% of their total lean leg mass after 10 days in bed (Kortebein et al. in JAMA, 297, 1772-1774, 2007). An increase in physical activity along with increasing dietary DHA/EPA may help gain independence and reduce the risk of falls and fractures as population ages.

Diabetes promotes aging. According to the World Health Organization (WHO), diabetes affects over 220 million people globally and the consequences of high blood sugar kill 3.4 million every year. The WHO is predicting deaths to double between 2005 and 2030. Type-2 diabetes typically affects adults, although overweight children can also develop it. Increased blood levels of ω-3 fatty acids from nonmarine sources [α-linolenic acid (ALA)] have been associated with reduced risk of type-2 diabetes (Brostow et al. in J. Am. Clin. Nutr. 94, 520-526, 2011). According to Feskens, E. J. M. in J. Am. Clin. Nutr. 94, 369-370 (2011), α-linolenic acid (ALA), the plant oil ω-3 fatty acid, was associated with a reduction in the risk of diabetes (21% reduction).

Food fortification is a practical approach for increasing the intake of ω-3 EPA/DHA. The difficulties encountered when fortifying foods with ω-3 EPA/DHA are primarily due to the lack of oxidative stability of these polyunsaturated fatty acids (PUFAs). The ω-3 PUFAs present in algae and fish oils are readily oxidized to produce rancidity or off-flavor volatiles when exposed to air, light, elevated temperatures, and/or transition metals (Harris, W. S. in Br. J. Nutr. 97, 593-595, 2007). Therefore, in terms of stability, preventing or reducing oxidation is at the forefront. The most common strategies to minimize oxidation potential of the ω-3 PUFAs are decreasing the amount of unsaturated bonds in such oils by the process of hydrogenation or the addition of natural or synthetic compounds with antioxidant properties. Hydrogenation creates trans double bonds which are known to contribute to heart disease. As to the use of antioxidants, Rosemary is one of the most widely used spices for its antioxidant properties. Tocopherols (vitamin E), which are natural constituents of most vegetable oils, serve as antioxidants to retard rancidity and as sources of the essential nutrient vitamin E. Synthetic antioxidants such as BHA, BHT, propyl gallate and TBHQ have traditionally been used to prevent oils from going rancid. However, the use of synthetic antioxidants is restricted by the Food and Drug Administration (FDA) because of food safety concerns.

Lactoferrin, an iron-binding glycoprotein of the transferrin family present in milk and other biological fluids, has been shown to modulate mucosal immunity, antitumor activity, and intestinal iron absorption (Weinberg, E. D. in Expert Opin. Invest. Drugs 12, 841-850, 2003; Parodi, P. W. in Current Pharmaceutical Design, 13, 813-828, 2007). Lactoferrin also exhibits antioxidant and antimicrobial activity (Shimazaki, K. and Watanabe, S. in The bio-defensive dairy food, Shimazaki, K. and Otani, H, eds., pp. 19-46. India. 2002). Bovine lactoferrin has been shown to reduce visceral fat in Japanese men and women with abdominal obesity (Ono et al. in J. Nutr. 104, 1688-1695, 2010). A decrease in lean mass and an increase in fat mass is often associated with aging. Physical activity is the likely candidate to prevent this undesirable change. The intake of high-quality protein for muscle growth and bovine lactoferrin for the control of visceral fat accumulation would be useful ways to maximize the beneficial effects of physical activity. Bovine lactoferrin may also help to prevent and even possibly reverse osteoporosis in postmenopausal women by stimulating osteoblastic bone formation (Bharadwaj et al. in Osteoporos. Int. 20, 1603-1611, 2009). Over 10 million women are afflicted with the age-related loss in bone density called osteoporosis.

Oral administration of bovine lactoferrin, and its role as a biopharmaceutical delivery system in the gastrointestinal tract, has been clearly established in research laboratories and in several experimental trials worldwide (Tomita et al. in Biochimie 91, 52-57, 2009). However, to commercialize bovine lactoferrin as a biopharmaceutical delivery system for human health applications requires a technology compatible with large-scale manufacturing practices. Such technology transfer must ensure the highest standards of product safety, quality assurance and delivery of an optimal dosage for an effective clinical outcome. There are five major issues critical for the commercialization of lactoferrin as a biopharmaceutical delivery system in humans including bioactivity, microbiological quality, endotoxin content, dosage, and stability in storage and in vivo.

As many proteins of biopharmaceutical interest, lactoferrin activity depends on the three-dimensional or tertiary structure of the molecule. Environmental conditions such as the presence of metals (iron, in particular), anionic ions (bicarbonate, in particular), salts, pH, temperature and conductivity are known to affect the biological activity of lactoferrin. In addition, protein isolation and processing conditions including storage, freezing/thawing, ultra-high-temperature (UHT) heating, and spray-drying could also adversely affect lactoferrin biological activity. Therefore, lactoferrin could partially or totally lose its biological activity during large-scale manufacturing and/or processing.

The microbiological quality of lactoferrin could significantly compromise the human health applications of commercial lactoferrin. In this context, lactoferrin isolated from dairy sources including colostrum, milk, whey and milk serum from cows, goats, buffalos, and sheep contains specific milk-borne human pathogens in particular Gram positive microflora and spore-forming organisms. Lactoferrin isolated from dairy sources may also be a carrier of bovine spongiform encephalopathy (BSE) agent, a pathogen which resists normal pasteurization (72° C., 120 s). Therefore, maintenance of lactoferrin stability during heat treatment at a substantially neutral pH and about 90° C. with assayable biological activity is of special importance for the production of food and pharmaceutical preparations. The term “substantially neutral pH” refers to a pH of between about 6.0 and about 8.0. In certain embodiments, lactoferrin is stabilized at a pH of between about 6.0 and about 7.0 or at a pH of about 6.3. The term “about”, in the context of pH, includes a pH that is ±0.1 pH unit from the recited value(s). The term “about”, in the context of temperature, includes temperatures that are ±3° C. of a recited temperature.

With respect to endotoxin content, the FDA requires that each dose have less than 0.5 endotoxin units (EU) for each ml of drug solution with a maximum tolerated limit of 5 EU for each kg of body weight. Endotoxins or lipopolysaccharides (LPS) are the outer membrane components of Gram negative bacteria. Endotoxins stimulate the production of cytokines and other mediators of inflammation, which in turn trigger a broad range of adverse physiological responses. Free radicals are reactive molecules with an unpaired electron; they are important mediators of cellular injury during endotoxemia, either as a result of molecular damage or by interfering with extracellular and intracellular regulatory processes. In addition, nitric oxide is thought to play a key role in the pathogenesis of endotoxic shock. Gram-negative bacteria present in milk and whey used in the isolation of lactoferrin and the processing plant environment contribute to the endotoxin levels in lactoferrin. Non-bacterial endototoxins, particularly 1,3-β-D-glucan from mold cell walls occurs in different environments such as chromatographic resins, processing equipment, and the water used in lactoferrin isolation. All of which could limit the use of lactoferrin isolated from dairy sources in food and pharmaceutical applications.

Knowledge of the dose-response or dose effect relationship is critical for choosing an optimum regimen for patients. In the simplest case, dose is directly proportional to the concentration of a drug or biopharmaceutical in the blood and at the site of action, but biological variability makes this assumption very tenuous. Hence knowledge of the concentration response usually provides better information. Therefore, lactoferrin dosage is highly critical in the development of a biopharmaceutical delivery system. A Continuing Survey of Food Intakes by Individuals (CSFII) sponsored by the United States Department of Agriculture (USDA) and conducted from 1994-1996 reveals that the average intake of milk and milk products in children 1 to 2 years old and teens 13 to 19 years old is about 396 g milk/day and 377 g milk/day, respectively. Taking into account that bovine milk contains 0.1 mg/ml to 0.2 mg/ml of lactoferrin, this is equivalent to 38 to 40 mg lactoferrin/day, respectively. Adults (20+) consume less milk, 240 g/day and their intake of lactoferrin is equal to about 24 mg/day. The consumption of lactoferrin for milk consumers in the 90^{th} percentile averages 73 mg/d for children, 75 mg/day for teens and 50 mg/day for adults.

Biopharmaceutical compositions should be stable in storage and in vivo. Protein and peptide formulations contain excipients to stabilize protein activity and reduce inactivation or loss due to adsorption to the container, oxidation, or hydrolysis. In some cases-insulin, for example-divalent cations such as Zn²⁺ are added to increase the duration of insulin effect. While formulation of proteins in solution or suspension are less costly to produce, not all therapeutic proteins can be stored in solution or suspensions, even when refrigerated (4° C.) or frozen (−20+ C). In those cases, freeze-dried formulations of proteins may be used as an alternative. Freeze-drying or lyophilization typically produces an amorphous form of protein that can be readily rehydrated or resuspended in water just prior to use. Protein lyophilization does not always yield increased stability compared with frozen liquid formulations. The stability of freeze-dried protein formulations can be improved by controlling moisture content and pH, and adding cryostabilizers or protectants such as sucrose, polyols, surfactants, and polymers.

Consumers, although concerned with the nutritional aspects, are probably most influenced by the flavor of the product. Light exposure, especially to wavelengths below 500 nm causes the destruction of light-sensitive bioactives (ω-3 fatty acids, CoQ10, xanthophylls), induces chemical reactions that affect proteins, and results in the development of unpleasant flavor in foods. Changes in flavor can be caused by the destruction of xanthophyll carotenoid pigments, protein breakdown, lipid hydrolysis, microbial spoilage or the destruction of highly polyunsaturated fatty acids such DHA and EPA. Off-flavors may thus be linked to a drop in the nutritional value of foods.

Many foods can be categorized as oil-in-water (O/W) emulsions, which consist of small lipid droplets dispersed in an aqueous medium e.g., milk, ice cream, salad dressings. In addition many medical foods and pharmaceuticals exist as this type of emulsion. One of the major mechanisms of oxidation of emulsified lipids is the iron-promoted degradation of lipid hydroperoxides into free radicals that can oxidize unsaturated fatty acids. Food and pharmaceutical emulsions typically contain ample endogenous concentrations of both iron and lipid hydroperoxides for this reaction to cause quality degradation (McClements, D. J. in Food Emulsions: Principles, Practices, and Techniques, pp. 95-174. CRC Press. Boca Raton, Fla., USA. 2005). The antioxidant activity of bovine lactoferrin in O/W emulsions depends on the lipid system, buffer, lactoferrin concentration, the presence of metal ions, and oxidation time (Huang et al. in J. Agric. Food Chem. 47, 1356-1361, 1999). The technological feasibility of formulating foods, medical foods, and pharmaceuticals with bovine lactoferrin and typical pH values in the range of 6.0 to 7.4, involves significant degradation of such a milk whey protein during thermal processing (Uzzan et al. in J. Food Sci. 72, E109-E114, 2007; Jacobsen et al. in Trends in Food Sci and Technol. 19, 76-93, 2008). Moreover, protein stability at elevated temperatures is a requirement for long-storage stability.

The present inventor has found that lactoferrin can be thermally stabilized in an effective and convenient manner and be palatable. It is therefore an object of the invention to provide heat-stabilized lactoferrin compositions that are both efficacious and palatable to the consumer. It is an also an object of the present invention to provide liquid compositions of heat-stabilized lactoferrin that are pH stable during the shelf life of the composition. Another object of the invention is to provide a heat-stabilized lactoferrin formulation which acts as a delivery system for anti-aging agents such as ω-3 fatty acids, CoQ₁₀, and xanthophylls. Other advantages include the ability of the heat-stabilized lactoferrin formulation to augment BCG vaccine efficacy and β-lactoglobulin used in the form of microcarriers as vehicles for the delivery of hydrophobic nutraceuticals (e.g., ω-3 fatty acids) and their protection from oxidizing agents. These and other objects of the present invention will become readily apparent from the detailed description which follows.

### BRIEF SUMMARY OF THE INVENTION

Heat resistant glycoprotein particles such as those of the glycoprotein lactoferrin for use as antioxidants and/or delivery systems of anti-aging agents i.e., ω-3 fatty acids, CoQ₁₀, xanthophylls in foods, medical foods, and pharmaceuticals having a pH of about 6.0 are produced by a process comprising dispersing the glycoprotein particles in a buffered water containing a zinc salt at a temperature of about 90° C. to about 150° C. Heat and storage stability of the glycoprotein lactoferrin particles can be dramatically improved with the addition of the branched-chain amino acids L-leucine, L-isoleucine, and L-valine at a 2:1:1 ratio. The glycoprotein lactoferrin particles alone or in combination with the branched-chain amino acids may be subsequently freeze-dried or lyophilized to remove the water from the glycoprotein lactoferrin particles. Another recommended method of protecting the glycoprotein lactoferrin particles from environmental stressors (heat, light, oxygen) when dispersed with ω-3 fatty acids, CoQ₁₀, and/or xanthophylls are accomplished through microencapsulation with polysaccharides. Microencapsulation with polysaccharides could reduce the allergenicity of proteins. The binding of polysaccharides to proteins could block allergic reactions by interfering with recognition sites on proteins that cause elevation of IgE.

It is also possible to provide protection of hydrophobic nutraceuticals i.e., DHA/EPA, CoQ₁₀, xanthophylls from oxidizing agents in aqueous medium by entrapping them within β-lactoglobulin microcarriers. The β-lactoglobulin microcarriers of the present invention exhibit potent antioxidant activity in O/W emulsions fortified with ω-3 DHA/EPA. The β-lactoglobulin microcarriers can also be conjugated with polysaccharides to improve the bioavailability of hydrobophic nutraceuticals and reduce the immunogenicity of β-lactoglobulin.

Methods for making the β-lactoglobulin microcarriers include the step of combining whey β-lactoglobulin with L-arginine base powder USP in buffered solution (10 mM potassium acetate, pH 6.0) followed by heating at 90° C. for 7 min, and chilling at 4° C. for 10 min.

### BRIEF DESCRIPTION OF THE DRAWINGS

FIG. 1 is a photograph illustrating a lactoferrin buffered solution (10 mM potassium acetate, pH 6.3) at 1 mg/ml after heated at 90° C. for 30 s to give a turbid solution and a heat-stabilized lactoferrin buffered solution (10 mM potassium acetate, pH 6.3) at 1 mg/ml prepared according to Example after heated at 90° C. for 30 s to give a transparent solution.

FIG. 2 demonstrates the enhanced storage stability of the heat-stabilized lactoferrin prepared according to Example 1 following incubation at 21° C. for 120 days.

FIG. 3 demonstrates the enhanced oxidative stability of the Menhaden oil/Smart Balance Omega oil-based, O/W emulsions containing the heat-stabilized lactoferrin prepared according to Example 4 following incubation at 30° C. for 32 days.

FIG. 4 demonstrates the enhanced storage stability of the delivery system containing CoQ₁₀ prepared according to Example 5 following incubation at 30° C. for 8 weeks.

FIG. 5 demonstrates the enhanced photo-stability of the delivery system containing lutein prepared according to Example 6 following incubation at 21° C. for 8 weeks.

FIG. 6 demonstrates the important role of the delivery system containing CoQ₁₀ prepared according to Example 5 in protecting cellular membranes from peroxidation damage caused by the increased metabolic demands of exercising muscles.

FIG. 7 demonstrates the important role of the delivery system containing CoQ₁₀ prepared according to Example 5 in attenuating the plasma GSH increase during exercise and recovery.

FIG. 8 demonstrates the important role of the delivery system containing CoQ₁₀ prepared according to Example 5 in attenuating the plasma CoQ₁₀ increase during exercise and recovery.

FIG. 9 demonstrates the important role of the delivery system containing CoQ₁₀ prepared according to Example 5 in enhancing cardioprotection at constant workload.

FIG. 10 demonstrates the IL-2 production in adult goats naturally infected by Corynebacterium pseudotuberculosis induced by the heat-stabilized lactoferrin prepared according to Example 1 with and without BCG.

### DETAILED DISCLOSURE OF THE INVENTION

The present invention relates to compositions for delaying aging. These compositions comprise: a lactoferrin; an acetate salt; a zinc salt; a branched-chain amino acid mix; a polysaccharide; a blend of ω-3, ω-6, and ω-9 fatty acids; anti-aging agents for additional antioxidant support in vivo; natural antioxidants for preventing lipid oxidation of ω-3 fatty acids, and other ingredients.

In certain embodiments, the components of the compositions are pharmaceutically acceptable. As used herein, a “pharmaceutically-acceptable” component is one that is suitable for use with humans and/or other animals without undue adverse side effects such as toxicity, irritation and allergic response commensurate with a reasonably benefit/risk ratio. The components for use in the present compositions, and the preferred amounts to be utilized, are described in detail hereinafter.

Lactoferrin

The compositions of the present invention contain an effective amount of lactoferrin, preferably, lactoferrin from cow's milk, as a therapeutically effective antioxidant. The lactoferrin to be used in the present invention can be also derived from human's milk, buffalo's milk, goat's milk, sheep's milk or the like. Lactoferrin typically comprises from about 0.01 to about 0.5%, and preferably from about 0.01 to about 0.1% by weight of the present compositions.

Metal Acetate Salt

The compositions of the present invention also comprise from about 0.01% to about 0.1%, preferably from about 0.01 to about 0.05%, of a metal acetate salt, preferably sodium or potassium, most preferably potassium acetate. This acetate salt acts to buffer the present compositions. Other pharmaceutically acceptable buffers (e.g., phosphate buffer) could not provide heat resistance to the zinc-saturated lactoferrin of the present invention.

Zinc Salt

The compositions of the present invention also contain from about 0.01 to about 0.1%, preferably from about 0.01 to about 0.05% of a zinc salt. The zinc salt used can be any of the forms commonly used such as ascorbate, aspartate, citrate, chloride, gluconate, lactate, sulfate, orotate, and oxide. It has been found, however, that the lactate salt is particularly preferred. Without being bound by theory, it is believed that the zinc cation inhibits thermal aggregation and inactivation of lactoferrin while the lactate anion magnifies the thermal resistance of lactoferrin.

Branched-Chain Amino Acid Mix

The compositions of the present invention may also contain an effective amount of a branched-chain amino acid mix as a therapeutically effective anti-aging. The branched-chain amino acids L-leucine, L-isoleucine, and L-valine may be used at a 2:1:1 ratio.

The ratio of the branched-chain amino acid mix to lactoferrin is generally chosen so that the final product is a formulation which besides the abovementioned ingredients contains 0.01% to 1.0% by weight of the branched-chain amino acid mix. The branched-chain amino acids used in the compositions of the present invention should be ‘free’ of added phospholipids (e.g., soy lecithin), and they are incorporated into the formulations containing the heat-stabilized lactoferrin of the present invention individually in amounts within the ranges indicated above resulting in a ‘matrix’ that readily retains the solvent components, and thereby preventing separation of the solvent from other components of the matrix.

The ‘matrix ‘comprising heat-stabilized lactoferrin alone and in combination with the branched-chain amino acids provides for minimized degradation of the anti-aging agents CoQ₁₀ and/or xanthophylls dispersed therein during storage under conditions that are known to accelerate the degradation of such anti-aging agents. In accordance with the present invention, therefore, degradation of CoQ₁₀ during storage for 8 weeks at 30° C. was observed to be about 30%. Light-sensitive degradation of xanthophyll carotenoid pigments (e.g., lutein) exposed to the damaging effect of UV and visible light for 12 h daily at 21° C. over 8 weeks was observed to be about 70%.

Anti-Aging Agents for Additional Antioxidant Support In Vivo

The present compositions may contain anti-aging agents such as CoQ₁₀, xanthophylls and/or L-glutathione which provide additional antioxidant support in vivo. CoQ₁₀, is made naturally in the body. Synthesis decreases progressively in humans above age 21. Therefore, CoQ₁₀ supplementation appears to be a means for older people to obtain their daily need of this nutrient. In addition to acting as an antioxidant, CoQ₁₀ increases oxygen use at the cellular level, improving the function of heart muscle cells and boosting capacity for aerobic exercise. CoQ₁₀ typically comprises from about 0.05% to about 0.4%, and preferably from about 0.05% to about 0.3% by weight of the present compositions. Lutein and zeaxanthin, xanthophyll carotenoid pigments found in spinach, kale, collards and broccoli, help prevent age-related macular degeneration (AMD) and cataracts (Ravikrishnan et al. in Food Chem. Toxicol. 49, 2841-2848, 2011). Lutein inhibits phospholipid peroxidation in human erythrocytes (Nakagawa et al. in Br. J. Nutr. 102, 1280-1284, 2009). In addition, lutein works to enhance the body's immune system. Our immune system weakens as we age, making us more susceptible to infections and cancer and slowing our healing responses. Furthermore, lutein can prompt an increase in physical activity in older people and make exercise more enjoyable. Lutein typically comprises from about 0.001% to about 0.1%, and preferably from about 0.001% to about 0.05% by weight of the present compositions. Zeaxanthin typically comprises from about 0.001% to about 0.1%, and preferably from about 0.001% to about 0.05% by weight of the present composition.

Astaxanthin, a xanthophyll carotenoid pigment found in marine animals, has recently received attention for its potent antioxidant activity (Higuera-Ciapara et al. in Crit. Rev. Food Sci. Nutr. 46, 185-196, 2006; Hussein et al. in J. Nat. Pro. 69, 443-449, 2006; Nakagawa et al. in Br. J. Nutr. 105, 1563-1571, 2011). Astaxanthin typically comprises from about 0.001% to about 0.1%, and preferably from about 0.001% to about 0.05% by weight of the present compositions. Fucoxanthin, a xanthophyll carotenoid pigment found in edible seaweeds, also has radical scavenging activity. However, fucoxanthin has been recently reported to break apart the stored coupled proteins within the fat cell and effectively turn the stored fat back into protein to be used as energy. Studies conducted on fucoxanthin-fed mice indicate that fucoxanthin has the ability to oxidize fat and release energy by adaptive thermogenesis within white adipose tissue (WAT) fat cells. WAT weight significantly decreased and the tissue-specific mitochondrial uncoupling protein 1 (UCP1) was expressed in the WAT, while there was no difference in WAT and little expression of UCP1 in the glycolipids-fed mice. The results indicate that fucoxanthin up regulates the expression of UCP1 in WAT, which may contribute to reducing WAT weight and abdominal fats. Diets containing fucoxanthin in combination with the heat-stabilized lactoferrin of Example 1 can prevent obesity (Maeda et al. in Int. J. Mol. Med. 18, 147-152, 2006; Maeda et al. in Asia Pac. J. Clin. Nutr. 17, 196-199, 2008; Ono et al. in J. Nutr. 104, 1688-1695, 2010). Fucoxanthin typically comprises from about 0.001% to about 0.1%, and preferably from about 0.001% to about 0.05% by weight of the present compositions. The CoQ₁₀ or xanthophyll carotenoid pigment is dispersed in emulsified lipid carriers which provide an effective barrier against environmental stresses (light, heat, oxygen).

Glutationine (GSH) is the most abundant low molecular weight thiol-containing compound in cells and a strong free radical scavenger. In its reduced form GSH protects against various oxidants, free radicals and cytotoxic agents. Adequate intracellular levels of GSH are necessary for T-lymphocyte activation. Factors that reduce intracellular GSH levels are alcohol, drinking, stress, pollution, toxins, cigarette smoking, and aging. A decrease in intracellular GSH levels leads to aging health declines. Reduced GSH is produced primarily in the liver (about 8-10 grams daily in adults) and is distributed to other body tissues through the bloodstream. In many health conditions, the demand for GSH exceeds the production. Supplementation with L-glutatione is recommended to increase body levels. L-glutathione typically comprises from about 0.1% to about 5%, and preferably from about 0.1% to about 1% by weight of the present compositions.

Antioxidants for Preventing Lipid Oxidation of ω-3 Fatty Acids

To increase the stability of ω-3 fatty acids (e.g., DHA, EPA) against oxidative degradation, it is advantageous to add stabilizers such as tocopherols (vitamin E) and Rosemary extracts. Tocopherols typically comprise from about 0.01 to about 0.03% and preferably from about 0.01% to about 0.02%. Rosemary extracts typically comprise from about 0.1% to about 0.4%, and preferably from about 0.1% to about 0.2%.

Ascorbic acid, ascorbyl palmitate and a phospholipid may be incorporated in combination with CoQ₁₀ to provide synergistic protection of ω-3 fatty acids from oxidation. It is preferred to use soy and sunflower phospholipids (lecithins) or fractions thereof which are abundant and economical. Preferably, the composition comprises about 0.1% CoQ₁₀, about 0.05% ascorbic acid, about 0.05% ascorbyl palmitate, and about 0.05% phospholipids.

Emulsified Lipid Carriers

The present compositions may contain a non-ionic surface-active emulsifier selected from the group consisting of mono and diglycerides of the fatty acid oleic acid, polyglyceride esters of the fatty acid oleic acid, mono, di, and polyglyeride esters of the fatty acid oleic acid further esterified with a dibasic organic acid selected from the group consisting of citric and lactic acids, acetylated mono, di, and polyglyceride esters of the fatty acid oleic acid further esterified with a dibasic organic acid selected from the group consisting of citric and lactic acids, and sorbitan esters of the fatty acid oleic acid. The lipid carrier can, in addition, comprise oils having ω-3 fatty acids as their primary fatty acid source (e.g., fish oils). Fish oils are selected from the group consisting of oils derived from anchovy, herring, and menhaden. The fish oil may be blended with oils having ω-6 and ω-9 fatty acids as their primary fatty acid source. The oils having ω-6 and ω-9 fatty acids are selected from canola, soybean, and olive. The non-ionic emulsifier comprises from about 0.1% to about 1.0%, and preferably from about 0.1% to about 0.5% by weight of the present compositions. The lipid comprises from about 0.5% to about 10%, and preferably from about 0.5% to about 1% by weight of the present compositions.

Other food- and pharmaceutical-grade emulsifiers, in particular the polysorbates (e.g., Tween 20, Tween 80), are undesirable in combination with the zinc-saturated lactoferrin of Example 1. One issue in using Tweens in protein preparations is their potential adverse effect on protein stability. One of the adverse effects is the oxidative damage of the residual peroxides in Tweens, which are generated through an autoxidation process during processing and storage. This can be a serious problem as proteins are generally sensitive to oxidative degradation and often formulated at relative low concentrations (Wang et al. in Int. J. Pharm. 347, 31-38, 2008).

β-Lactoglobulin Microcarriers

The compositions of the present invention may contain β-lactoglobulin microcarriers. The β-lactoglobulin microcarriers are designed for the delivery of hydrophobic nutraceuticals (e.g., ω-3 fatty acids) and their protection against oxidizing agents. Whey β-lactoglobulin is combined with L-arginine base powder USP and heated at a temperature of 90° C. for 7 min or longer. The elevated temperature in the presence of L-arginine base powder USP enhances the antioxidative activity of β-lactoglobulin. Immunogenicity of β-lactoglobulin can be decreased by conjugation with polysaccharides. L-arginine has been shown to enhance host defense mechanisms (Morris, S. M., Jr. in Br. J. Pharmacol. 157, 922-930, 2009). Preferably, the composition in the β-lactoglobulin microcarriers comprise about 1% β-lactoglobulin and about 0.5% L-arginine base powder USP. The hydrophobic nutraceuticals ω-3 fatty acids, CoQ₁₀, and xanthophylls can be added to the β-lactoglobulin microcarriers in an amount of from about 0.01% to about 1.0% by weight.

As analyzed by gas chromatography (GC), the percentages of retained ω-3 DHA and EPA in O/W emulsions prepared with menhaden oil/Smart Blend Omega oil (0.5% w/w, based on the weight of the emulsion) at 1:1 ratio and entrapped within the β-lactoglobulin microcarriers were 79.5% and 61.8%, respectively, after 28 days of storage at 30° C.

Polysaccharides

The compositions of the present invention may contain a polysaccharide selected from the group consisting of alginates, carrageenans, dextrans, galactomanans, glucomanans, kefiran, pectin, and starch. The polysaccharide comprises from about 0.1% to about 2%, and preferably from about 0.1% to about 1% by weight of the present compositions.

Sweetening Agents

The compositions of the present invention may contain a heat-stable sweetener selected from the group consisting of sucralose, acesulfame potassium, and sugar alcohols (e.g., erythritol, D-mannitol, sorbitol). The heat-stable sweetener comprises from about 0.1% to about 10%, and preferably from about 0.1% to about 1% by weight of the present compositions.

Method of Treatment

The compositions of the present invention additionally relate to a method for delaying aging. The method of treatment herein comprises orally administering to a human or lower animal in need of such treatment a safe and effective amount of a liquid anti-aging composition according to the present invention.

The term “safe and effective amount”, as used herein, means a quantity of the lactoferrin-containing liquid composition sufficient to yield the desired anti-aging efficacy without undue adverse side effects (endotoxin toxicity, allergic response) commensurate with a reasonable benefit/risk ratio when used in the manner of this invention. The specific safe and effective amount will, obviously, vary with such factors as the particular condition, the duration of the treatment, the physical condition of the patient, the nature of concurrent therapy (if any), and the specific formulation and optional components employed. However, a patient in need of such an anti-aging treatment will typically receive, for example, from about 100 mg to about 300 mg of heat-stabilized lactoferrin, 100 mg to about 1000 mg of branched-chain amino acids, 100 mg to about 1000 mg of the ω-3fatty acids DHA/EPA, 100 mg to about 300 mg of CoQ₁₀, 5 mg to about 50 mg of xanthophylls, 100 mg to about 1000 mg of L-Arginine, and 100 mg to about 1000 mg of L-Glutathione daily.

The subject application also provides the following non-limiting embodiments:

1. A composition comprising:

(a) heat stabilized lactoferrin, heat stabilized lactoglobulin or a combination of heat stabilized lactoferrin and heat stabilized lactoglobulin;

(b) a buffer selected from the group consisting of acetate salts of:
i. sodium,
ii. potassium,
iii. magnesium, and
iv. calcium; and

(c) optionally, one or more anti-aging agent selected from the group consisting of:
i. coenzyme Q₁₀;
ii. a xanthophyll, such as astaxanthin, fucoxanthin and/or zeaxanthin; and
iii. L-Glutathione; and
iv. leutein.

2. The composition according to embodiment 1, wherein said composition comprises heat stabilized lactoferrin.

3. The composition according to embodiment 2, wherein said heat-stabilized lactoferrin has been mixed with zinc lactate and said heat stabilized lactoferrin is saturated with zinc.

4. The composition according to embodiment 1, wherein said composition comprises heat stabilized lactoglobulin or heat stabilized lactoferrin.

5. The composition according to embodiment 4, wherein said heat stabilized lactoglobulin comprises a mixture of L-arginine and lactoglobulin and/or said heat stabilized lactoferrin comprises a combination of branched chain amino acids and beta-lactoferrin.

6. The composition according to any one of embodiments 1-5, said composition further comprising a combination of branched chain amino acids.

7. The composition according to embodiment 6, said wherein said branched chain amino acids are L-leucine, L-isoleucine and L-valine.

8. The composition according to embodiment 7, wherein said amino acids are present at a ratio of 2:1:1 (L-leucine:L-isoleucine:L-valine).

9. The composition according to embodiment 2, said composition further comprising a combination of branched chain amino acids.

10. The composition according to embodiment 9, said wherein said branched chain amino acids are L-leucine, L-isoleucine and L-valine.

11. The composition according to embodiment 10, wherein said amino acids are present at a ratio of 2:1:1 (L-leucine:L-isoleucine:L-valine).

12. The composition according to any one of embodiments 1-11, said composition further comprising a nonionic surface-active emulsifier selected from the group consisting of:
a. mono and diglycerides of the fatty acid oleic acid;
b. polyglyceride esters of the fatty acid oleic acid;
c. mono, di, and polyglyceride esters of the fatty acid oleic acid further esterified with a dibasic organic acid selected from the group consisting of citric and lactic acids;
d. acetylated mono, di, and polyglyceride esters of the fatty acid oleic acid further esterified with a dibasic organic acid selected from the group consisting of citric and lactic acids;
e. sorbitan esters of the fatty acid oleic acid; and
f. any combination of a-e.

13. The composition of any one of embodiments 1-12, said composition further comprising a natural antioxidant selected from the group consisting of:
a. Rosemary extracts;
b. tocopherols;
c. ascorbic acid;
d. ascorbyl palmitate;
e. phospholipids; and
f. combinations thereof.

14. The composition according to any one of embodiments 1-14, said composition further comprising a heat-stable sweetener selected from the group consisting of:
a. sucralose;
b. acesulfame potassium; and
c. sugar alcohols.

15. The composition according to any one of embodiments 1-14, said composition further comprising an edible oil blend comprising ω-3, ω-6 and ω-9 fatty acids.

16. The composition of any one of embodiments 1-15, said composition comprising one or more anti-aging agent selected from the group consisting of:
a. coenzyme Q₁₀;
b. a xanthophyll, such as astaxanthin, fucoxanthin and/or zeaxanthin; and
c. L-Glutathione; and
d. leutein.

17. The composition of any one of embodiments 1-17, said composition further comprising a polysaccharide selected from the group consisting of:
a. alginates;
b. carrageenans;
c. dextrans;
d. galactomannans;
e. glucomanans;
f. kefiran;
g. pectin;
h. starch; and
i. combinations thereof.

18. The composition according to embodiment 16, wherein said anti-aging agent is encapsulated with a polysaccharide selected from the group consisting of:
a. alginates;
b. carrageenans;
c. dextrans;
d. galactomannans;
e. glucomanans;
f. kefiran;
g. pectin;
h. starch; and
i) combinations thereof.

19. The composition according to embodiment 1, said composition comprising:
(a) heat stabilized lactoferrin, heat stabilized lactoglobulin or a combination of heat stabilized lactoferrin and heat stabilized lactoglobulin;
(b) a buffer selected from the group consisting of acetate salts of:
   i. sodium,
   ii. potassium,
   iii. magnesium, and
   iv. calcium; and
(c) one or more anti-aging agent selected from the group consisting of:
   i. coenzyme Q₁₀;
   ii. a xanthophyll, such as astaxanthin, fucoxanthin and/or zeaxanthin; and
   iii. L-Glutathione;
   iv. leutein; and
(d) a nonionic surface-active emulsifier selected from the group consisting of:
   i. mono and diglycerides of the fatty acid oleic acid;
   ii. polyglyceride esters of the fatty acid oleic acid;
   iii. mono, di, and polyglyceride esters of the fatty acid oleic acid further esterified with a dibasic organic acid selected from the group consisting of citric and lactic acids;
   iv. acetylated mono, di, and polyglyceride esters of the fatty acid oleic acid further esterified with a dibasic organic acid selected from the group consisting of citric and lactic acids;
   v. sorbitan esters of the fatty acid oleic acid; and
   vi. any combination of a-e.

20. The composition according to embodiment 19, said composition further comprising:
(a) a natural antioxidant selected from the group consisting of:
   i. Rosemary extracts;
   ii. tocopherols;
   iii. ascorbic acid;
   iv. ascorbyl palmitate;
   v. phospholipids; and
   vi. combinations thereof;
(b) an edible oil blend comprising ω-3, ω-6 and ω-9 fatty acids; and
(c) a polysaccharide selected from the group consisting of:
   i. alginates;
   ii. carrageenans;
   iii. dextrans;
   iv. galactomannans;
   v. glucomanans;
   vi. kefiran;
   vii. pectin;
   viii. starch; and
   ix) combinations thereof

21. The composition according to embodiments 19-20, said composition further comprising a heat-stable sweetener selected from the group consisting of:
a. sucralose;
b. acesulfame potassium; and
c. sugar alcohols.

22. The composition according to any one of embodiments 19-21, said composition further comprising a combination of branched chain amino acids.

23. The composition according to embodiment 22, said wherein said branched chain amino acids are L-leucine, L-isoleucine and L-valine.

24. The composition according to embodiment 23, wherein said amino acids are present at a ratio of 2:1:1 (L-leucine:L-isoleucine:L-valine).

25. A method of preparing heat stabilized lactoferrin comprising heating a mixture of lactoferrin, zinc lactate and, optionally, branched-chain amino acids to a temperature between about 90° C. and about 150° C. for a period of time, said mixture having a pH of about 6.0 to about 8.0. In certain embodiments, the mixture can be heated for a period of about 30 seconds to less than 5 minutes, about 30 seconds to less than sixty minutes or a period of about 30 seconds to one day (or more).

26. The method according to embodiment 25, wherein said mixture contains branched chain amino acids, said branched chain amino acids comprising L-leucine, L-isoleucine and L-valine.

27. The method according to embodiment 26, wherein said amino acids are present at a ratio of 2:1:1 (L-leucine:L-isoleucine:L-valine).

28. A method for stabilizing an anti-aging agent comprising emulsifying one or more anti-aging agent to a composition comprising:
(a) heat stabilized lactoferrin, heat stabilized lactoglobulin or a combination of heat stabilized lactoferrin and heat stabilized lactoglobulin;
(b) a buffer selected from the group consisting of acetate salts of:
   i. sodium,
   ii. potassium,
   iii. magnesium, and
   iv. calcium; and
(c) a nonionic surface-active emulsifier selected from the group consisting of:
   i. mono and diglycerides of the fatty acid oleic acid;
   ii. polyglyceride esters of the fatty acid oleic acid;
   iii. mono, di, and polyglyceride esters of the fatty acid oleic acid further esterified with a dibasic organic acid selected from the group consisting of citric and lactic acids;
   iv. acetylated mono, di, and polyglyceride esters of the fatty acid oleic acid further esterified with a dibasic organic acid selected from the group consisting of citric and lactic acids;
   v. sorbitan esters of the fatty acid oleic acid; and
   vi. any combination of a-e.

29. The method according to embodiment 28, wherein said one or more anti-aging agent is selected from the group consisting of:
a. coenzyme Q₁₀;
b. a xanthophyll, such as astaxanthin, fucoxanthin and/or zeaxanthin; and
c. L-Glutathione; and
d. leutein.

30. The method according to embodiments 28-29, wherein said anti-aging agent is encapsulated in a polysaccharide selected from the group consisting of:
i. alginates;
ii. carrageenans;
iii. dextrans;
iv. galactomannans;
v. glucomanans;
vi. kefiran;
vii. pectin;
viii. starch; and
ix) combinations thereof.

31. A method for delivering an anti-aging agent to a human or animal, said method comprising administering a therapeutically effective amount of a composition according to embodiments 1-24 to a human or animal.

32. A method of immunizing a human or animal comprising administering a composition comprising heat stabilized lactoferrin and a vaccine to human or animal.

33. The method according to embodiment 32, wherein the production of IL-2 is increased in said human or animal.

Additional embodiments provided by the subject invention include:

1. A composition exhibiting antioxidant activity for protection against oxidative damage at the cellular level comprising:

(a) branched-chain amino acids and whey protein;

(b) one or more anti-aging agent selected from the group consisting of:
i. coenzyme Q₁₀;
ii. xanthophylls such as lutein, astaxanthin, fucoxanthin and/or zeaxanthin; and
iii. L-Glutathione;

2. The composition according to embodiment 1, wherein said composition comprises branched-chain amino acids;

3. The composition according to embodiment 2, wherein said branched-chain amino acids are L-Leucine, L-Isoleucine and L-Valine; 4. The compositions of embodiment 3, wherein said L-Leucine, L-Isoleucine and L-Valine are present at a ratio of 2:1:1;

5. The compositions according to embodiment 1, wherein said the whey protein is lactoferrin;

6. The composition of embodiment 5, wherein said the lactoferrin is heat-stabilized;

7. The composition of embodiment 1, wherein said the whey protein is β-lactoglobulin;

8. The composition according to any one of embodiments 1-7, said composition further comprising a buffer selected from the group consisting of acetate salts of:

a. sodium,

b. potassium,

c. magnesium,

d. calcium; and

e. any combination of a-d;

9. The composition according to any one of embodiments 1-8, said composition further comprising a nonionic surface-active emulsifier selected from the group consisting of:

a. mono and diglycerides of the fatty acid oleic acid,

b. polyglyceride esters of the fatty acid oleic acid,

c. mono, di, and polyglyceride esters of the fatty acid oleic acid further esterified with a dibasic organic acid selected from the group consisting of citric and lactic acids,

d. acetylated mono, di, and polyglyceride esters of the fatty acid oleic acid further esterified with a dibasic organic acid selected from the group consisting of citric and lactic acids,

e. sorbitan esters of the fatty acid oleic acid; and

f. any combination of a-e;

10. The composition of according to any one of embodiments 1-9, said composition further comprising a natural antioxidant selected from the group consisting of:

a. Rosemary extracts,

b. tocopherols,

c. ascorbic acid,

d. ascorbyl palmitate,

e. phospholipids; and

f. any combination of a-e;

11. The composition according to any one of embodiments 1-10, said composition further comprising a heat-stable sweetener selected from the group consisting of:

a. sucralose,

b. acesulfame potassium,

c. sugar alcohols; and

d. any combination of a-c;

12. The composition according to any one of embodiments 1-11, said composition further comprising an edible oil blend consisting of ω-3, ω-6 and ω-9 fatty acids;

13. The composition according to any one of embodiments 1-12, said composition further comprising a polysaccharide selected from the group consisting of:

a. alginates,

b. carrageenans,

c. dextrans,

d. galactomanans,

e. glucomanans,

f. kefiran,

g. pectin,

h. starch; and

i. any combination of a-h;

14. The composition of embodiment 1, wherein said the heat-stabilized lactoferrin comprises a mixture of lactoferrin, natural zinc lactate, and optionally, branched-chain amino acids;

15. A method to stabilize lactoferrin comprising combining lactoferrin zinc lactate and branched-chain amino acids at a substantially neutral pH and heating the composition to about 90° C.;

16. A method to form stable β-lactoglobulin microcarriers comprising combining β-lactoglobulin and L-arginine and heating the composition to a temperature of about 90° C.;

17. A method for delaying aging in humans or animals, said method comprising administering a therapeutically effective amount of the anti-aging agents of embodiment 1, wherein said the anti-aging agents are capable of delaying aging in humans or animals;

18. A method to augment vaccine efficacy against infections comprising administering a composition comprising heat-stabilized lactoferrin according to any one of embodiments 1-14 and a vaccine to a subject (human or animal) that is to be immunized;

19. The method according to embodiment 18, wherein said heat-stabilized lactoferrin is a composition according to embodiment 14;

20. A method for delaying aging in humans or animals, said method comprising administering a therapeutically effective amount of the composition of embodiment 1, wherein said the composition of embodiment 1 is capable of delaying aging in humans or animals by augmenting vaccine efficacy against infections;

All patents, patent applications, provisional applications, and publications referred to or cited herein are incorporated by reference in their entirety, including all figures and tables, to the extent they are not inconsistent with the explicit teachings of this specification.

The following examples further demonstrate and describe embodiments with the scope of the present invention. The examples are given solely for the purpose of illustration and are not to be constructed as a limitation of the present invention as many variations thereof as possible without departing from the spirit and scope.

### EXAMPLE 1

### Preparation of Heat-Stabilized Lactoferrin

The heat-stabilized lactoferrin of the present invention is prepared as follows: bovine lactoferrin (0.1 grams) is added to 100 ml of a buffered solution (10 mM potassium acetate, pH 6.3) and allowed to dissolve by stirring at low speed. To the buffered solution containing bovine lactoferrin, a small amount of zinc lactate (0.01 grams) is added, followed by stirring at low speed for few minutes. It should be noted here that the bovine lactoferrin sample needs to be exposed to air in the presence of zinc ions. The carbon dioxide (CO₂) from the air is converted to bicarbonate ions so that it can participate in the reaction. Bicarbonate is required for the ligation of zinc ions to bovine lactoferrin. The branched-chain amino acids L-leucine, L-isoleucine, and L-valine at a 2:1:1 ratio (0.2 grams) may be alternatively used in combination with zinc lactate to stop heat-induced degradation of lactoferrin, which is responsible for the loss of biological activity of heated lactoferrin at a substantially neutral pH and UHT conditions.

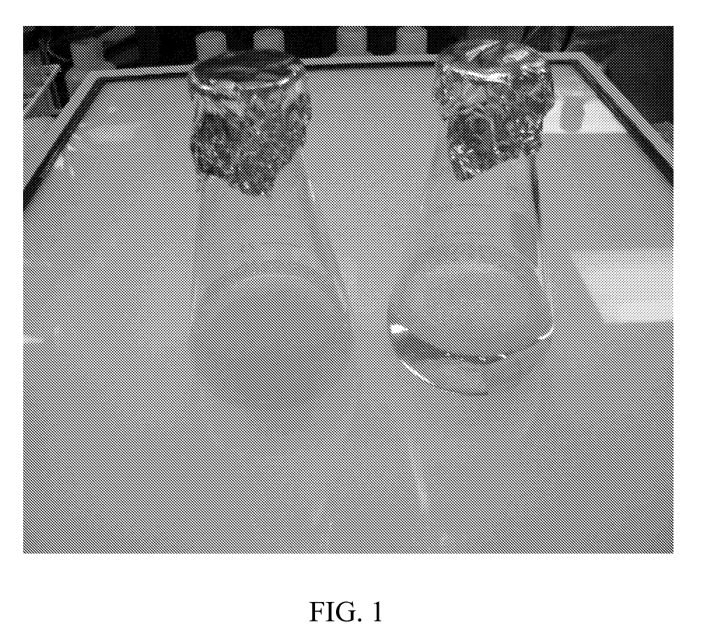

Formulations containing heat-stabilized lactoferrin (1 mg/ml) without added branched-chain amino acids (Formulation 1) and heat-stabilized lactoferrin (1 mg/ml) with added branched-chain amino acids (2 mg/ml) (Formulation 2) were dissolved in the buffered solution (10 mM potassium acetate, pH 6.3) to make 4 liters solution. The two liquid formulations were heated by using a plate heat exchanger at 90° C. for 30 seconds, and cooled to 5° C., after which they were stored in sterilized bottles, and bubbled with carbon dioxide (CO₂) in an aseptic room. The two resultant liquid formulations with a zinc ion concentration of 2.4 mg per 100 ml had excellent taste and appearance. As shown in FIG. 1, a lactoferrin buffered solution (10 mM potassium acetate, pH 6.3) at 1 mg/ml becomes turbid i.e., the formation of protein aggregates when heated under these conditions. However, the heat-stabilized lactoferrin buffered solution (10 mM potassium acetate, pH 6.3) at 1 mg/ml without added branched chain amino acids (Formulation 1) showed no turbidity after heating to give a transparent solution. The addition of the branched chain amino acids L-leucine, L-isoleucine, and L-valine at a 2:1:1 ratio to the heat-stabilized lactoferrin (Formulation 2) gave a more viscous liquid but the transparency of the liquid was maintained. Lactoferrin aggregation occurred at pH 6.3, which corresponded to its high thermal instability.

### EXAMPLE 2

### Radial Immunodifusion Quantitation of Heat-Stabilized Lactoferrin

Quantitative analysis of the two carbonated formulations containing the heat-stabilized lactoferrin prepared as in Example 1 was performed by a Single Radial Immunodifusion (SRID) kit (Cardiotech Services, Louisville, Ky., USA). No extraction process was required because the kit is especially designed to measure bovine lactoferrin. Potassium acetate-buffered solutions (10 mM, pH 6.3) containing the zinc-saturated lactoferrin (1 mg/ml) were diluted 1:2 with double deionized water in order to be in the concentration range of the kit (250 to 1000 μg/ml). Five μl of the diluted sample was accurately and carefully pipetted into the sampling well of the immunodifusion gel plate. The plate was covered and kept in a desiccator over water that was placed in a 37° C. incubator for 48 hours. The heat-stabilized lactoferrin of Example 1 diffuses through the gel while reacting with bovine lactoferrin antibodies, thus producing a clear halo circle which area is correlated with bovine lactoferrin concentration. The diameter of the precipitin ring around each sample was measured to determine the quantity of zinc-saturated lactoferrin present in each formulation and results are presented in Table 1.

TABLE 1
Radial immunodifusion quantitation of formulations
containing heat-stabilized lactoferrin

| | |
|---|---|
| Formulation type | Lactoferrin (μg/ml) |

Formulation 1

| | |
|---|---|
| Unheated | 1,000 |
| Heated | 997 |

Formulation 2

| | |
|---|---|
| Unheated | 1,000 |
| Heated | 999 |

The results given in Table 1 show clearly that the two formulations prepared with the heat-stabilized lactoferrin of the present invention having a zinc ion concentration of 2.4 mg/100 ml withstands heat treatment at 90° C. for 30 seconds. Bovine lactoferrin loses its immunological properties when it is heat-treated at 63 and 85° C. (Uzzan et al. in J. Food Sci. E109-E114, 2007).

### EXAMPLE 3

### Storage Stability of Heat-Stabilized Lactoferrin

To test the storage stability of the formulations containing the heat-stabilized lactoferrin prepared as in Example 1, one ml of Formulation 1 or Formulation 2 was transferred to a 1.5-ml centrifuge tube and centrifuged at 3000 rpm for 10 min. Sampling was conducted over storage time at 21° C. and lactoferrin concentration determined by means of high-pressure liquid chromatography (HPLC) according to methods described by Palmano, K. P. and Elgar, D. F. in J. Chromatogr. 947, 307-311 (2002). Remaining rates of heat-stabilized lactoferrin were calculated according to the following formula:

$$Remaining rate of lactoferrin ( \% ) = \frac{lactoferrin concentration after storage}{lactoferrin concentration immediately after preparation} \times 100$$

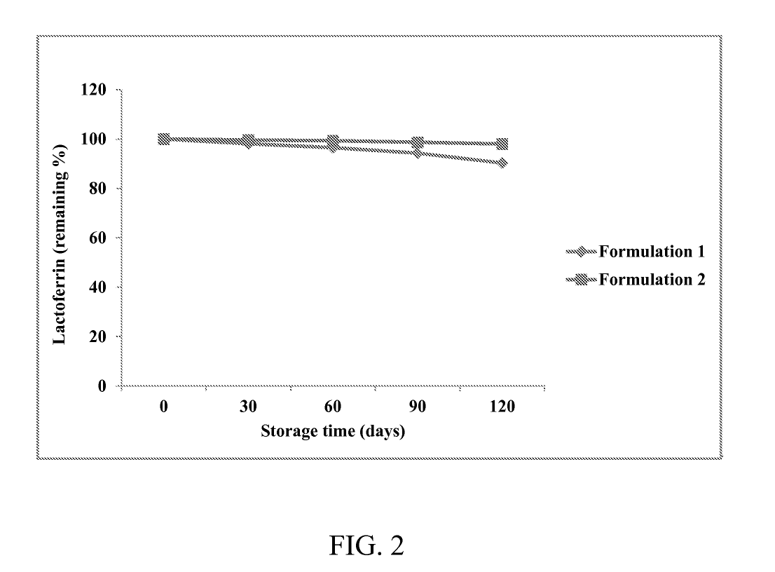

The concentration of heat-stabilized lactoferrin remaining in the supernatant is shown in FIG. 2. As shown in FIG. 2, the two formulations with a zinc ion concentration of 2.4 mg/100 ml exhibit 90% or higher of the remaining rate of lactoferrin after 120 day-storage at 21° C. A higher stabilizing effect was observed in the formulation with added branched-chain amino acids (Formulation 2).

### EXAMPLE 4

### Antioxidant Activity of Heat-Stabilized Lactoferrin

Carbonated formulations containing the heat-stabilized lactoferrin without added branched-chain amino acids (Formulation 1) and with added branched-chain amino acids (Formulation 2) prepared as in Example 1 could become a healthy alternative in soft drink machines. Carbonated O/W Menhaden oil-based emulsions ‘blended’ with Smart Balance Omega. sup.™ oil and stabilized against oxidation with heat-stabilized lactoferrin without added branched-chain amino acids (Formulation 1) and with added branched-chain amino acids (Formulation 2) prepared as in Example 1 stayed fresh up to 4 months when refrigerated. The Smart Balance Omega. sup.™ contains good amounts of tocopherols (also good antioxidants). Individual isomers ranged as follows: α, 55.4 mcg/g; β, 175 mcg/g; and δ, 84.5 mcg/g.

We bubbled carbon dioxide (CO₂) gas through five O/W emulsions prepared as follows: A. Control. Menhaden oil/Smart Blend Omega oil at 1:1 ratio (0.5% w/w, based on the weight of the emulsion) is homogenized with triglycerol monooleate (0.1% w/w, based on the weight of the oil). An aqueous solution (99.5% of double deionized water) containing potassium acetate (0.01% w/w, based on the weight of the emulsion). The pH of this aqueous solution is adjusted to 6.3 with 0.1 N potassium hydroxide (KOH). The emulsified oil blend (0.5%) is added to the aqueous solution (99.5%) and homogenized for 3 min using a hand-held homogenizer. The coarse emulsion was then homogenized (72° C., 22.5 MPa) and pasteurized (72° C., 120 s). B. Formulation 1. Menhaden oil/Smart Blend Omega oil at 1:1 ratio (0.5% w/w, based on the weight of the emulsion) is homogenized with triglycerol monooleate (0.1% w/w, based on the weight of the oil). An aqueous solution (99.5% w/w of double deionized water, based on the weight of the emulsion) containing potassium acetate (0.01% w/w, based on the weight of the emulsion) and the heat-stabilized lactoferrin (0.1% w/w, based on the weight of the emulsion) without added branched-chain amino acids of Example 1. The pH of this aqueous solution is adjusted to 6.3 with 0.1 N potassium hydroxide (KOH). This aqueous solution had already been heated at 90° C. for 30 seconds. The emulsified oil blend (0.5%) is added to the aqueous solution (99.5%) and homogenized for 3 min using a hand-held homogenizer. The coarse emulsion was then homogenized (72° C., 22.5 MPa) and pasteurized (72° C., 120 s). C. Formulation 2. Menhaden oil/Smart Blend Omega oil at 1:1 ratio (0.5% w/w, based on the weight of the emulsion) is homogenized with triglycerol monooleate (0.1% w/w, based on the weight of the oil). An aqueous solution (99.5% w/w of double deionized water, based on the weight of the emulsion) containing potassium acetate (0.01% w/w, based on the weight of the emulsion), and the heat-stabilized lactoferrin (0.1% w/w, based on the weight of the emulsion) with added branched-chain amino acids (0.2% w/w, based on the weight of the emulsion) of Example 1. The pH of this aqueous solution is adjusted to 6.3 with 0.1 N potassium hydroxide (KOH). This aqueous solution had already been heated at 90° C. for 30 seconds. The emulsified oil blend (0.5%) is added to the aqueous solution (99.5%) and homogenized for 3 min with a hand-held homogenizer. This coarse emulsion was then homogenized (72° C., 22.5 MPa) and pasteurized (72° C., 120 s). D. Formulation 3. Menhaden oil/Smart Blend Omega oil at 1:1 ratio (0.5%) is homogenized with triglycerol monooleate (0.1% w/w, based on the weight of the oil), then Rosemary Oxy'Block™ (0.4% w/w, based on the weight of the oil) is added to this oil blend followed by homogenization. An aqueous solution (99.5% w/w of double deionized water, based on the weight of the emulsion) containing potassium acetate (0.01% w/w, based on the weight of the emulsion), and the heat-stabilized lactoferrin (0.1% w/w, based on the weight of the emulsion) without added branched-chain amino acids of Example 1. The pH of this aqueous solution is adjusted to 6.3 with 0.1 N potassium hydroxide (KOH). This aqueous solution had already been heated at 90° C. for 30 seconds. The emulsified oil blend (0.5%) is added to the aqueous solution (99.5%) and homogenized for 3 min using a hand-held homogenizer. The coarse emulsion was then homogenized (72° C., 22.5 MPa) and pasteurized (72° C., 120 s). E. Formulation 4. Menhaden oil/Smart Blend Omega oil at 1:1 ratio (0.5% w/w, based on the weight of the emulsion) is homogenized with triglycerol monooleate (0.1% w/w, based on the weight of the oil), then Rosemary Oxy'Block. sup.™ (0.4% w/w, based on the weight of the oil) is added to this oil blend followed by homogenization. An aqueous solution (99.5% w/w of double deionized water, based on the weight of the emulsion) buffered with potassium acetate (0.01% w/w, based on the weight of the emulsion), and the heat-stabilized lactoferrin (0.1% w/w, based on the weight of the emulsion) with added branched-chain amino acids (0.2% w/w, based on the weight of the emulsion) of Example 1. The pH of this aqueous solution is adjusted to 6.3 with 0.1 N potassium hydroxide (KOH). This aqueous solution had already been heated at 90° C. for 30 seconds. The emulsified oil blend (0.5%) is added to the aqueous solution (99.5%) and homogenized for 3 min with a hand-held homogenizer. The coarse emulsion was then homogenized (72° C., 22.5 MPa) and pasteurized (72° C., 120 s).

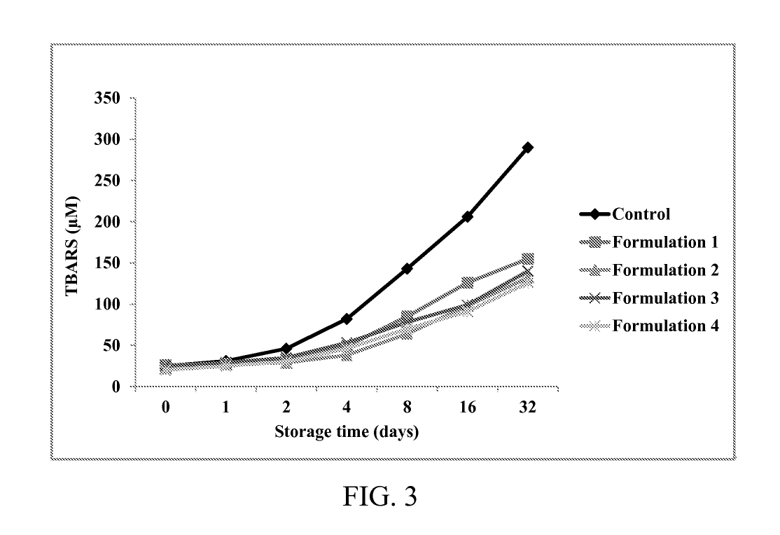

The samples (40 ml each) were aseptically dispensed into 50-ml capped, sterile brown glass bottles, bubbled with carbon dioxide (CO₂), and stored in an incubator for 32 days at 30° C. Samples were taken at 0, 1, 2, 4, 8, 16, and 32 days for analysis. The samples were examined for visual appearance and odor. Triplicate samples of the inventive and control O/W emulsions were analyzed for thiobarbituric acid reactive substances (TBARS) according to methods described by Nanua et al. in J. Dairy Sci. 83, 2426-2431 (2000). TBARS are a group of lipid oxidation products that react with thiobarbituric acid (TBA) to form colored products. Although malonaldehyde (MDA) is used as the standard, other compounds also react with TBA to give colored pigments. The antioxidant activity of the heat-stabilized lactoferrin of Example 1 is demonstrated in FIG. 3.

### EXAMPLE 5

### Delivery System for COQ-HD 10

A delivery system containing the anti-aging agent CoQ₁₀ is prepared as follows: A. Encapsulated-1 (CoQ₁₀). Primary Emulsion. An aqueous emulsifier solution containing the heat-stabilized lactoferrin (0.2% w/w, based on the weight of the emulsion) without added branched-chain amino acids of Example 1 in potassium acetate buffer (10 mM, pH 6.3) was stirred for 2 h to ensure dissolution. Menhaden oil/Smart Blend Omega oil at 1:1 ratio (1% w/w, based on the weight of the emulsion) containing microcrystalline CoQ₁₀ (20%, based on the weight of the oil) and Rosemary Oxy'Block. sup.™ (0.4% w/w, based on the weight of the oil) was added to the heat-stabilized lactoferrin solution without added branched-chain amino acids so that the emulsion system contained 1% Menhaden oil/Smart Blend Omega oil, 0.2% heat-stabilized lactoferrin, 0.2% CoQ₁₀, and 98.6% buffer (w/w). This mixture was coarsely homogenized for 3 min using a hand blender. Secondary Emulsion. An aqueous solution containing high-methoxyl pectin (0.2% w/w, based on the weight of the emulsion) in potassium acetate buffer (10 mM, pH 6.3) was stirred for 3 h to ensure dissolution.

The primary and secondary emulsions were blended at 1:1 ratio. The final emulsion system contained 0.5% Menhaden oil/Smart Blend Omega oil, 0.1% heat-stabilized lactoferrin, 0.1% CoQ₁₀ and 0.1% high-methoxyl pectin (w/w). The zinc ion concentration after dilution became 2.4 mg/100 ml. This delivery system for CoQ₁₀ was homogenized (72° C., 22.5 MPa), followed by pasteurization (72° C., 120 s). B. Encapsulated-2 (CoQ₁₀). Primary Emulsion. An aqueous emulsifier solution containing the heat-stabilized lactoferrin (0.2% w/w, based on the weight of the emulsion) with added branched-chain amino acids (0.4% w/w, based on the weight of the emulsion) of Example 1 in potassium acetate buffer (10 mM, pH 6.3) was stirred for 2 h to ensure dissolution. Menhaden oil/Smart Blend Omega oil at 1:1 ratio (1% w/w, based on the weight of the emulsion) containing microcrystalline CoQ₁₀ (20% w/w, based on the weight of the oil) and Rosemary Oxy'Block™ (0.4% w/w, based on the weight of the oil) was added to the heat-stabilized lactoferrin solution with added branched-chain amino acids so that the emulsion system contained 1% Menhaden oil/Smart Blend Omega oil, 0.2% heat-stabilized lactoferrin, 0.4% branched-chain amino acids, 0.2% CoQ₁₀ and 98.2% buffer (w/w). This mixture was coarsely homogenized for 3 min using a hand blender. Secondary Emulsion. An aqueous solution containing high-methoxyl pectin (0.2% w/w, based on the weight of the emulsion) in potassium acetate buffer (10 mM, pH 6.3) was stirred for 3 h to ensure dissolution. The primary and secondary emulsions were blended at 1:1 ratio. The final emulsion system contained 0.5% Menhaden oil/Smart Blend Omega oil, 0.1% heat-stabilized lactoferrin, 0.2% branched-chain amino acids, 0.1% CoQ₁₀, and 0.1% high-methoxyl pectin (w/w). The zinc ion concentration after dilution became 2.4 mg/100 ml. This delivery system for CoQ₁₀ was homogenized (72° C., 22.5 MPa), followed by pasteurization (72° C., 120 s). C. Unencapsulated (CoQ₁₀). Menhaden oil/Smart Blend Omega oil at 1:1 ratio (0.5% w/w, based on the weight of the emulsion), microcrystalline CoQ₁₀ (20% w/w, based on the weight of the oil), and Tween-20 (0.1% w/w, based on the weight of the emulsion) was added to an aqueous solution buffered with potassium acetate (10 mM, pH 6.3) so that the final emulsion system contained 0.5% Menhaden oil/Smart Blend Omega oil, 0.1% CoQ₁₀, and 0.1% Tween-20 (w/w). This mixture was homogenized (72° C., 22.5 MPa), followed by pasteurization (72° C., 120 s).

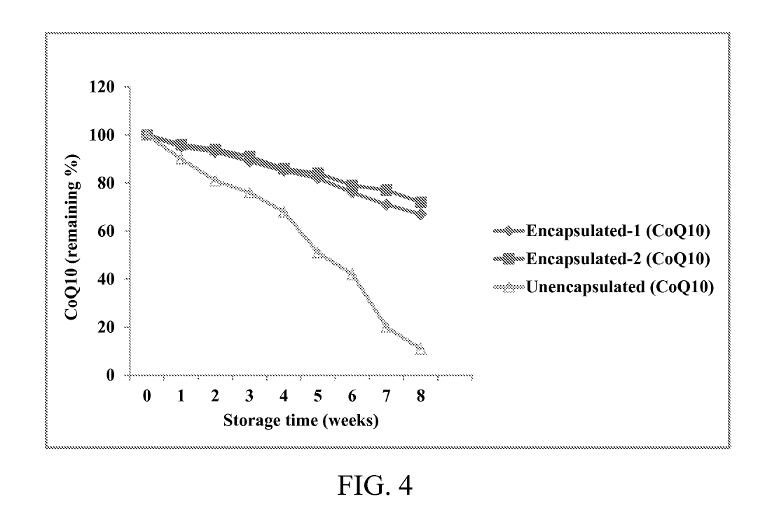

The samples (500 ml each) were aseptically dispensed into clean 600-ml bottles made from polyethylene terephthalate (PET) combined with UV absorbers (UV-PET). After being dispensed, the samples were bubbled with carbon dioxide (CO₂) gas and immediately transferred to an environmental chamber, held at a constant temperature of 30° C. Samples were taken at 0, 1, 2, 3, 4, 5, 6, 7, and 8 weeks for analysis. The samples were examined for visual appearance and odor. Triplicate samples of the inventive and control O/W emulsions were assayed for CoQ₁₀ using high-performance liquid chromatography (HPLC) according to methods described by Lunetta, S. and Roman, M. in J. AOAC Int. 9, 702-708 (2008). The delivery system of the present invention has been shown to vastly improve CoQ₁₀ stability by providing exceptional protection from heat as seen in FIG. 4.

### EXAMPLE 6

### Delivery System for Lutein

A delivery system containing the anti-aging agent lutein is prepared as follows:

A. Encapsulated-1 (Lutein). Primary Emulsion. An aqueous emulsifier solution containing the heat-stabilized lactoferrin (0.2% w/w, based on the weight of the emulsion) without added branched-chain amino acids of Example 1 in potassium acetate buffer (10 mM, pH 6.3) was stirred for 2 h to ensure dissolution. Menhaden oil/Smart Blend Omega oil at 1:1 ratio (1% w/w, based on the weight of the emulsion) containing microcrystalline lutein (20% w/w, based on the weight of the oil) and Rosemary Oxy'Block™ (0.4% w/w, based on the weight of the oil) was added to the heat-stabilized lactoferrin solution without added branched-chain amino acids so that the emulsion system contained 1% Menhaden oil/Smart Blend Omega oil, 0.2% heat-stabilized lactoferrin, 0.2% lutein, and 98.6% buffer (w/w). This mixture was coarsely homogenized for 3 min using a hand blender. Secondary Emulsion. An aqueous solution containing high-methoxyl pectin (0.2% w/w, based on the weight of the emulsion) in potassium acetate buffer (10 mM, pH 6.3) was stirred for 3 h to ensure dissolution.

The primary and secondary emulsions were blended at 1:1 ratio. The final emulsion system contained 0.5% Menhaden oil/Smart Blend Omega oil, 0.1% heat-stabilized lactoferrin, 0.1% lutein, and 0.1% high-methoxyl pectin (w/w). The zinc ion concentration after dilution became 2.4 mg/100 ml. This delivery system for lutein was homogenized (72° C., 22.5 MPa), followed by pasteurization (72° C., 120 s).

B. Encapsulated-2 (Lutein). Primary Emulsion. An aqueous emulsifier solution containing the heat-stabilized lactoferrin (0.2% w/w, based on the weight of the emulsion) with added branched-chain amino acids (0.4% w/w, based on the weight of the emulsion) of Example 1 in potassium acetate buffer (10 mM, pH 6.3) was stirred for 2 h to ensure dissolution. Menhaden oil/Smart Blend Omega oil at 1:1 ratio (1% w/w, based on the weight of the emulsion) containing microcrystalline lutein (20% w/w, based on the weight of the oil) and Rosemary Oxy'Block™ (0.4% w/w, based on the weight of the oil) was added to the heat-stabilized lactoferrin solution with added branched-chain amino acids so that the emulsion system contained 1% Menhaden oil/Smart Blend Omega oil, 0.2% heat-stabilized lactoferrin, 0.4% branched-chain amino acids, 0.2% lutein, and 98.2% buffer (w/w). This mixture was coarsely homogenized for 3 min using a hand blender. Secondary Emulsion. An aqueous solution containing high-methoxyl pectin (0.2% w/w, based on the weight of the emulsion) in potassium acetate buffer (10 mM, pH 6.3) was stirred for 3 h to ensure dissolution.

The primary and secondary emulsions were blended at 1:1 ratio. The final emulsion system contained 0.5% Menhaden oil/Smart Blend Omega oil, 0.1% heat-stabilized lactoferrin, 0.2% branched-chain amino acids, 0.1% lutein, and 0.1% high-methoxyl pectin (w/w). The zinc ion concentration after dilution became 2.4 mg/100 ml. This delivery system for lutein was homogenized (72° C., 22.5 MPa), followed by pasteurization (72° C., 120 s).

C. Unencapsulated (Lutein). Menhaden oil/Smart Blend Omega oil at 1:1 ratio (0.5% w/w, based on the weight of the emulsion), microcrystalline lutein (20% w/w, based on the weight of the oil), and Tween-20 (0.1% w/w, based on the weight of the emulsion) was added to an aqueous solution buffered with potassium acetate (10 mM, pH 6.3) so that the final emulsion system contained 0.5% Menhaden oil/Smart Blend Omega oil, 0.1% lutein, and 0.1% Tween-20 (w/w). This mixture was homogenized (72° C., 22.5 MPa), followed by pasteurization (72° C., 120 s).

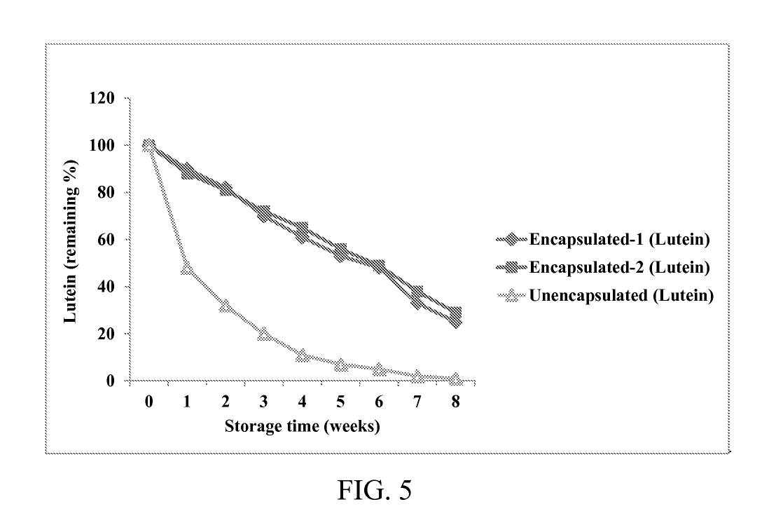

The samples (500 ml each) were aseptically dispensed into clean, clear 600-ml bottles made from polyethylene terephthalate (PET). After being dispensed, the samples were bubbled with carbon dioxide (CO₂) gas, transferred to an environmental chamber, and held at a constant temperature of 21° C. The lighting was provided by four 60 W cool white fluorescent tubes for 12 h daily. Samples were taken at 0, 1, 2, 3, 4, 5, 6, 7, and 8 weeks for analysis. The light was evenly distributed over all of the bottles within the chamber. The samples were examined for visual appearance and odor. Duplicate samples of the inventive and control O/W emulsions were assayed for lutein using high-pressure liquid chromatography-photodiode array detector-mass spectrometry detectors (HPLC-PDA-MS/MS) according to methods described by de Rosso, W. and Mercadante, A. Z. in J. Agric. Food Chem. 55, 5062-5072 (2007). The delivery system of the present invention has been shown to vastly improve lutein stability by providing exceptional protection from the damaging effect of UV/visible light as seen in FIG. 5.

### EXAMPLE 7

### Human Clinical Study

This study was undertaken to demonstrate that the intake of the two microencapsulated CoQ₁₀ formulations of Example 5 have clinical significance in modulating the rate of aging. The intake of the microencapsulated CoQ₁₀ formulations of Example 5 may prove to be a simple and straightforward means of attenuating the formation of free radicals, which are intimately linked to age-related pathology in older individuals.

Oxygen free radicals are metabolic products possessing at least one unpaired electron in their outer orbital shell. This unpaired electron makes the compound unstable, thereby increasing its potential reactivity with other molecules creating the possibility of damage to cell walls and cellular constituents, such as DNA. Excessive free radical generation has been observed from exercise and has been implicated in cellular and tissue injury, decreased muscle function, and prolonged recovery following exercise.

During free-radical stress, the oxidants act like invaders, taking away electrons from precious molecules at every turn. The antioxidants we normally produce in our bodies or add to our diets (such as vitamins C and E and the minerals selenium and zinc) help to cancel out the chemical activity of free radicals and protect our cells. Antioxidants surrender themselves, offering their electrons freely to neutralize the invading oxidants in these metabolic reactions.

Since the antioxidant activity of CoQ₁₀ is directly related to its energy carrier function, CoQ₁₀ molecules can generally undergo oxidation/reduction reactions and therefore can become powerful antioxidants. CoQ₁₀ becomes reduced as it accepts electrons as part of its work in the electron transport chain of oxidative phosphorylation (cellular energy production). And it becomes oxidized as it gives up electrons to pass them along the chain. In the reduced form, CoQ₁₀ can give up electrons quickly and easily, and thus acts as an antioxidant against free radicals. Since free radicals are highly reactive molecules with unpaired electrons, CoQ₁₀'s remarkable electron donor activity makes it an ideal antioxidant. It neutralizes the toxic effect of the free radical by giving it an electron and completing its lacking electron pair.

Since the electron-rich reduced form of CoQ₁₀, vitamin E, and other antioxidants support free-radical fighting defenses, their presence becomes vital in strategies to prevent free-radical damage and premature aging. Because the oxidized form of vitamin E can be reduced by CoQ₁₀, vitamin E recycling is enhanced. As a recycler of vitamin E, CoQ₁₀ makes its antioxidant partner more available to help trap free radicals before they do their damage.

### Methods:

The study Procedures and Assessments involved identifying and enrolling subjects, obtaining written informed consents, and randomization into one of three groups The subjects were 18 healthy, nonsmoking male volunteers, aged 30-35 years who had not taken any CoQ₁₀, vitamin supplements, or medication within the previous 4 weeks. Following a standardized 10-minute warm up period breathing room air, subjects (n=6 per group) ingested 250 ml of water containing placebo (5 g sucralose/acesulfame potassium) or 250 ml of a carbonated O/W emulsion containing either encapsulated-1 CoQ₁₀ (250 mg CoQ₁₀+131 mg ω-3 fatty acids EPA/DHA+250 mg lactoferrin+6 mg zinc+5 g sucralose/acesulfame potassium) produced by homogenization at 72° C. and 22.5 MPa, followed by pasteurization (72° C., 120 s) or encapsulated-2 CoQ₁₀ (250 mg CoQ₁₀+131 mg ω-3 fatty acids EPA/DHA+250 mg lactoferrin+6 mg zinc+500 mg branched-chain amino acids+5 g sucralose/acesulfame potassium) produced by homogenization at 72° C. and 22.5 MPa, followed by pasteurization (72° C., 120 s) . The Exercise consisted of cycling for 25 minutes at the determined lactate threshold of each subject under hypoxic conditions. The goal of the exercise protocol was to maximize oxygen flux through the tissues, yet to ensure an intensity of exercise that would not lead to acidosis that may cause exercise to terminate prematurely.

Hypoxia was induced by having subjects breathe a gas mixture of 16% oxygen and 84% nitrogen through a one-way valve. Subjects sat quietly for 60-minutes breathing room air following the 25-minute exercise protocol. A second dose of placebo or microencapsulated CoQ₁₀ formulation was administered following exercise.

A venous catheter was inserted into a forearm vein before each exercise session. Blood samples were collected 15 minutes prior to exercise, at 8, 16, and 24 minutes of exercise, and at 60 and 120 minutes after the start of exercise. Urine samples were collected 10-minutes before exercise and at 28, 46, 88, and 120 minutes after the start of exercise. Blood and urine samples were immediately frozen at −70° C. until analysis. Heart rate was recorded at 9, 17, and 25 minutes of exercise.

Blood plasma was analyzed for CoQ₁₀ concentration by the modified method of Vadhanavikit et al. in Anal. Biochem. 142, 155-158 (1984) and total glutathione concentration by the modified method of Jacobsen et al. in Clin. Chem. 40, 873-881 (1994). Urine was analyzed for malonaldehyde (MDA) using thiobarbituric acid according to methods described by Drury et al. in Clin. Chim Acta 263, 177-185 (1997). Heart rate was determined using a heart rate monitor (HRM USA, Inc., Warminster, Pa.).

### Results:

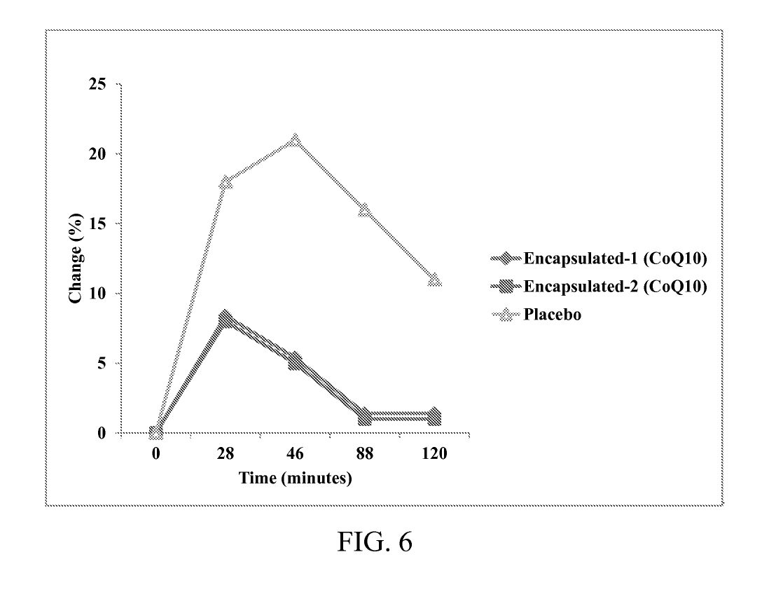

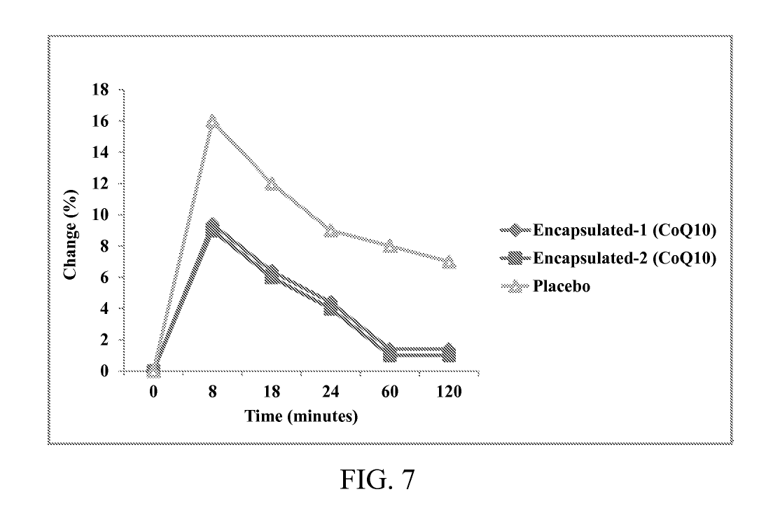

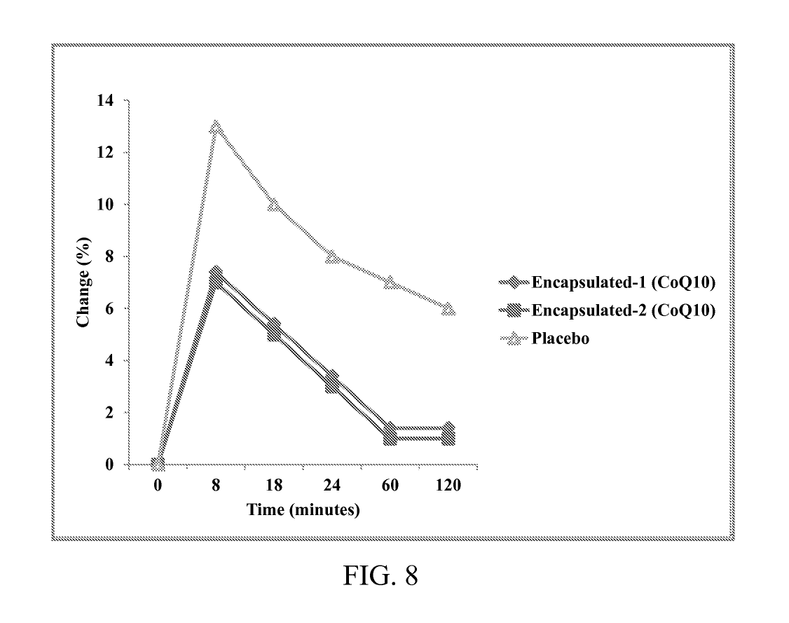

Measured urinary MDA levels increased significantly (p<0.05) during placebo, but remained lowered during CoQ₁₀ administration (FIG. 6). At the final collection point (120 minutes from the start of exercise) MDA was increased by 10% over baseline in the placebo group, but was maintained at baseline condition in the CoQ₁₀ groups. The maintenance of MDA near the baseline condition found in the CoQ₁₀ groups indicates that cellular membranes incurred little, if any, peroxidation damage compared to the placebo group. Blood plasma GSH and CoQ₁₀ increases during oxidative stress. The GSH and CoQ₁₀ increase were attenuated in the CoQ₁₀ groups during exercise and recovery suggesting that the designed CoQ₁₀ formulations represent a new potential system for oral delivery of CoQ₁₀ (FIG. 7 and FIG. 8, respectively). These positive effects may be also attributed to the fact that lactoferrin, elemental zinc, and/or branched-chain amino acids are the dominant ingredients present in the designed CoQ₁₀ formulation i.e., encapsulated-1 (CoQ₁₀) and encapsulated-2 (CoQ₁₀). Healthy individuals concerned with age management need a foundation program of antioxidant support. In this context, supplemental bovine lactoferrin, 100 mg for 7 days, followed by 200 mg of lactoferrin for 7 days, has been shown to support immune and antioxidant status in healthy human males (Mulder et al. in Nutr. Res. 28, 583-589 (2008). Zinc plays a fundamental role in antioxidant defense (Ho, E. in J. Nutr. Biochem. 15, 572-578, 2004). Branched-chain amino acid supplementation, 1.5 mg/g body weight/day in drinking water, has been shown to reduce oxidative damage in skeletal muscles and white adipose tissue of middle-aged mice (D'Antona et al. in Cell Metabolism 12, 362-372, 2010). And while each anti-aging agent (CoQ₁₀, lactoferrin, zinc, branched-chain amino acids) contributes immeasurably to the health of the cell, in combination they are unbeatable.

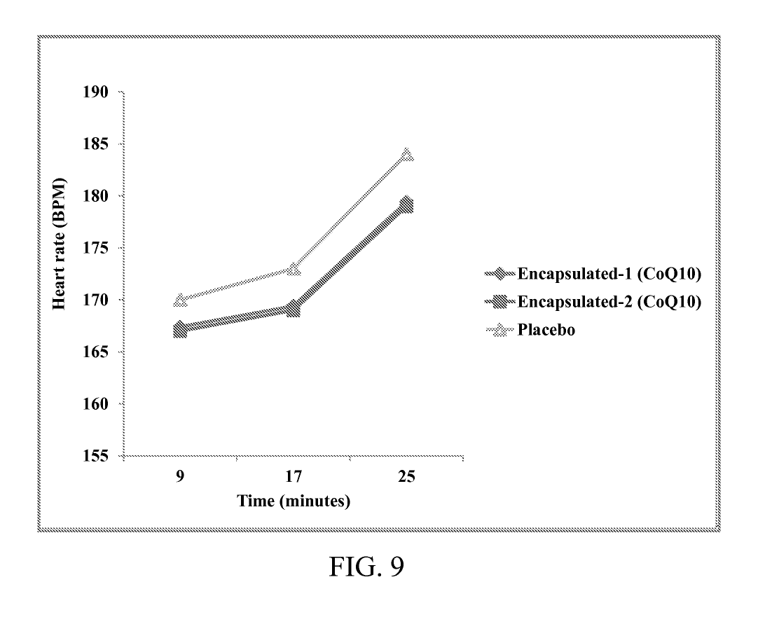

The results of this clinical study clearly indicate that the delivery system containing CoQ₁₀ described herein as encapsulated-1 (CoQ₁₀) and encapsulated-2 (CoQ₁₀) can attenuate free radical production during hypoxic exercise. Heart rate gradually increased through the first two collection times for the placebo and CoQ10 groups (FIG. 9). However, at the end of the 25 minute exercise period heart rate was lower for the CoQ10 groups than the placebo group suggesting enhanced cardioprotection. All results were statistically significant (p<0.05).

### EXAMPLE 8

### T-Helper Activity of Heat-Stabilized Lactoferrin

A well-established biomarker of ageing is the loss of the subcutaneous adipose skin layer, which in turn can lead to opportunistic pathologies associated with old age such as infections (Tomas-Loba et al. in Cell 135, 609-622, 2008). Caseous lymphadenitis (CLA), a disease of goats and sheep, is caused by Corynebacterium pseudotuberculosis. CLA is characterized by fibrous encapsulated abscesses in the peripheral lymph nodes and sometimes the lungs and other visceral organs. The progression of CLA in goats and sheep involves primary wound infection, lymphatic and hematogenous dissemination, and secondary infection of lymph nodes and various visceral organs. In horse and cattle, Corynebacterium pseudotuberculosis infection occurs following entry of the bacteria through skin wounds. The ability to control intracellular Corynebacterium pseudotuberculosis infection relies on cellular immunity and generation of a strong T-cell helper response. The widely used tuberculosis vaccine is a live attenuated strain of Mycobacterium bovis Bacillus Calmette-Guérin (BCG). However, the efficacy of BCG in generating a protective response against infectious diseases such as Mycobacterium tuberculosis and Corynebacterium pseudotuberculosis has failed. Presently, the model adjuvant used with BCG is complete Freund's adjuvant (CFA), which is highly toxic and not suitable for human use and becoming more undesirable for use in animals.

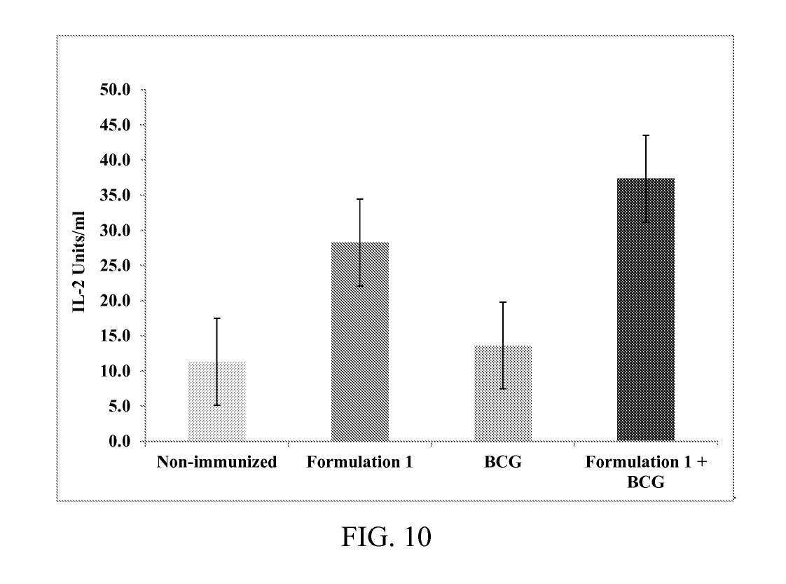

To determine the adjuvant efficacy of the heat-stabilized lactoferrin of Example 1 (Formulation 1) to boost BCG efficacy, 28 naturally infected goats (adults, 1-4 years old) were selected. Formulation 1 injection consists of heat-stabilized lactoferrin crystals dissolved in a sterile solution. Each 10 ml vial of Formulation 1 injection contains heat-stabilized lactoferrin 500 mg. Immunizations were performed using standard NIH protocols for evaluation of BCG vaccines, modified as follows: 7 goats were immunized per group with 2 ml of zinc-saturated lactoferrin (Formulation 1), once, subcutaneously (s.c.). All formulations of BCG with and without heat-sterilized lactoferrin utilized BCG at 2 ml/goat Peripheral blood mononuclear cells (PBMCs) were obtained from each immunization group. The PBMCs were cultivated under conditions which permit proliferation. Interleukin 2 (IL-2) production was measured in a bioassay. The results of different groups were compared for significant differences by Student's t-test (p<0.05). BCG immunization with heat-stabilized lactoferrin as adjuvant showed strong IL-2 production (p<0.05) indicating intact T-helper function (FIG. 10).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 5,098,722 March/1992 Tanaka et al.
U.S. Pat. No. 5,606,086 February /1997 Dosako et al.
U.S. Pat. No. 6,479,627 November /2002 Ota et al.
U.S. Pat. No. 6,500,472 December /2002 Uchida et al.
U.S. Pat. No. 6,673,382 January/2004 Kawama et al.
U.S. Pat. No. 6,864,231 March/2005 Chokshi
U.S. Pat. No. 7,326,775 February/2008 Naidu
20050271603 December/2005 Krammer et al.
Balagopal et al., “Effects of aging on in vivo synthesis of skeletal muscle myosin heavy-chain and sarcoplasmic protein in humans,” Am. J. Physiol. 273, E790-E800, 1997.
Bharadwaj et al., “Milk ribonuclease-enriched lactoferrin induces positive effects on bone turnover markers in postmenopausal women,” Osteoporos. Int. 20, 1603-1611, 2009.
Brostow et al., “Omega-3 fatty acids and incident type 2 diabetes: the Singapore Chinese health study,” J. Am. Clin. Nutr. 94, 520-526, 2011.
Charlton, K. E., “Eating well: ageing gracefully!,” Asia Pacific J. Clin. Nutr. 11, S607-S617, 2002.
D'Antona et al., “Branched-chain amino acid supplementation promotes survival and supports cardiac and skeletal muscle mitochondrial biogenesis in middle-aged mice,” Cell Metabolism 12, 362-372, 2010.
Damsgaard et al., “Fish oil supplementation modulates immune function in healthy infants,” J. Nutr. 137, 1031-1036, 2007.
De la Fuente, M., “Effects of antioxidants on immune system ageing,” Eur. J. Clin. Nutr. 56, 55-58, 2002.
de Rosso, W. and Mercadante, A. Z., “Identification and quantification of carotenoids, by HPLC-PDA-MS/MS, from Amazonian fruits,” J. Agric. Food Chem. 55, 5062-5072, 2007.
Drury et al., “Comparison of urinary and plasma manlondialdehyde in preterm infants,” Clin. Chim Acta 263, 177-185, 1997.
Feskens, E. J. M., “The prevention of type 2 diabetes: should we recommend vegetable oils instead of fatty fish?,”J. Am. Clin. Nutr. 94, 369-370, 2011.
Harman, D. “Aging: a theory based on free radical and radiation chemistry,” J. Geront. 11, 298-300, 1956.
Harris, W. S., “n-3 Fatty acid fortification opportunities and obstacles,” Br. J. Nutr. 97, 593-595, 2007.
Higuera-Ciapara et al., “Astaxanthin: a review of its chemistry and applications,” Crit. Rev. Food Sci. Nutr. 46, 185-196, 2006.
Ho, E., “Zinc deficiency, DNA damage and cancer risk,” J. Nutr. Biochem. 572-578, 2004.
Hoppe et al., “The use of whey or skimmed milk powder in fortified blended foods for vulnerable groups,” J. Nutr. 138, 145S-161S, 2008.
Huang et al., “Effect of lactoferrin on oxidative stability of corn oil emulsions and liposomes,” J. Agric. Food Chem. 47, 1356-1361, 1999.
Hussein et al., “Astaxanthin, a carotenoid with potential in human health and nutrition,” J. Nat. Prod. 69, 443-449, 2006.
Jacobsen et al., “Rapid HPLC determination of total homocysteine and other thiols in serum and plasma: sex differences and correlation with cobalamin and folate concentrations in healthy subjects,” Clin. Chem. 40, 873-881, 1994.
Jacobsen et al., “Antioxidant strategies for preventing deterioration of foods enriched with n-3 polyunsaturated lipids: a comparative evaluation,” Trends in Food Sci and Technol. 19, 76-93, 2008.
Kew et al., “Lack of effect of foods enriched with plant- or marine-derived n-3 fatty acids on human immune function,” Am. J. Clin. Nutr. 77, 1287-1295, 2003.
Kortebein et al., “Effect of 10 days of bed rest on skeletal muscle in healthy older adults,” JAMA, 297, 1772-1774, 2007.
Lunetta, S. and Roman, M., “Determination of coenzyme Q10 content in raw materials and dietary supplements by high-performance liquid chromatography-UV: collaborative study,” J. AOAC Int. 9, 702-708, 2008.
Maeda et al., “Fucoxanthin and its metabolite, fucoxanthinol, suppress adipocyte differentiation in 3T3-L1 cells,” Int. J. Mol. Med. 18, 147-152, 2006.
Maeda et al., “Seaweed carotenoid, fucoxanthin, as a multi-functional nutrient,” Asia Pac. J. Clin. Nutr. 17, 196-199, 2008.
McClements, D. J., “Emulsion ingredients,” Food Emulsions: Principles, Practices, and Techniques, pp. 95-174. CRC Press. Boca Raton, Fla., USA. 2005.
Morris, S. M., Jr., “Recent advances in arginine metabolism: roles and regulation of the arginases,” Br. J. Pharmacol. 157, 922-930, 2009.
Mulder et al., “Bovine lactoferrin supplementation supports immune and antioxidant status in healthy human males,” Nutr. Res. 28, 583-589, 2008.
Nakagawa et al., “Antioxidant effect of lutein towards phospholipid hydroperoxidation in human erythrocytes,” Br. J. Nutr. 102, 1280-1284, 2009.
Nakagawa et al., “Antioxidant effect of astaxanthin on phospholipid peroxidation in human erythrocytes,” Br. J. Nutr. 105, 1563-1571, 2011.
Nanua et al., “Influence of high-oryzanol rice bran oil on the oxidative stability of whole milk powder,” J. Dairy Sci. 83, 2426-2431, 2000.
Ono et al., “Potent anti-obesity effect of enteric-coated lactoferrin: decrease in visceral fat accumulation in Japanese men and women with abdominal obesity after 8-week administration of enteric-coated tables,” J. Nutr. 104, 1688-1695, 2010.
Palmano, K. P. and Elgar, D. F., “Detection and quantitation of lactoferrin in bovine whey samples by reversed-phase high performance liquid chromatography on polystyrene-divinylbenzene,” J. Chromatogr. 947, 307-311, 2002.
Parodi, P. W., “A role of milk proteins and their peptides in cancer prevention,” Current Pharmaceutical Design 13, 813-828, 2007.
Ravikrishnan et al., “Safety assessment of lutein and zeaxanthin (Lutemax 2020): subchronic toxicity and mutagenicity studies,” Food Chem. Toxicol. 49, 2841-2848, 2011.
Rooyackers et al., “Effect of age on in vivo rates of mithocondrial protein synthesis in human skeletal muscle,” Proc. Natl. Acad. Sci. USA 93, 15364-15369, 1996.
Smith et al., “Dietary omega-3 fatty acid supplementation increases the rate of muscle protein synthesis in older adults: a randomized controlled trial,” Am. J. Clin. Nutr. 93, 402-412, 2011.
Shimazaki, K. and Watanabe, S., “Biological significance of lactoferrin and lactoperoxidase in milk,” The bio-defensive dairy food, Shimazaki, K. and Otani, H, eds., pp. 19-46. India. 2002.
Short, K. R. and Nair, K. S., “ Mechanisms of sarcopenia of aging,” J. Endocrinol. Invest. 22, 95-105, 1999.
Short et al., “Decline in skeletal muscle mitochondrial function with aging in humans,” PNAS 102, 5618-5623, 2005.
Tomas-Loba et al., “Telomerase reverse transcriptase delays aging in cancer-resistant mice,” Cell 135, 609-622.
Tomita et al., “Twenty-five years of research on bovine lactoferrin applications,” Biochimie 91, 52-57, 2009.
Trebble et al., “Inhibition of tumour necrosis factor-alpha and interleukin 6 production by mononuclear cells following dietary fish-oil supplementation in healthy men and response to antioxidant co-supplementation,” Br. J. Nutr. 90, 405-412, 2003.
Uzzan et al., “Thermal and storage stability of nutraceuticals in a milk beverage dietary supplement,” J. Food Sci. 72, E109-E114, 2007.
Weinberg, E. D., “The therapeutic potential of lactoferrin,” Expert Opin. Invest. Drugs 12, 841-850, 2003.
Vadhanavikit et al., “Quantitative determination of coenzyme Q₁₀ in human blood for clinical studies,” Anal. Biochem. 142, 155-158, 1984.
Wang et al., “Dual effects of tween 80 on protein stability,” Int. J. Pharm. 347, 31-38, 2008.

1. A composition comprising:
(a) heat stabilized lactoferrin, heat stabilized lactoglobulin or a combination of heat stabilized lactoferrin and heat stabilized lactoglobulin;
(b) a buffer selected from the group consisting of acetate salts of:
i) sodium,
ii) potassium,
iii) magnesium, and
iv) calcium; and
(c) optionally, one or more anti-aging agent selected from the group consisting of:
i) coenzyme Q₁₀;
ii) a xanthophyll, such as astaxanthin, fucoxanthin and/or zeaxanthin; and
iii) L-Glutathione; and
iv) leutein.

2. The composition according to claim 1, wherein said composition comprises heat stabilized lactoferrin.

3. The composition according to claim 2, wherein said heat-stabilized lactoferrin has been mixed with zinc lactate and said heat stabilized lactoferrin is saturated with zinc.

4. The composition according to claim 1, wherein said composition comprises heat stabilized lactoglobulin or heat stabilized lactoferrin.

5. The composition according to claim 4, wherein said heat stabilized lactoglobulin comprises a mixture of L-arginine and lactoglobulin and/or said heat stabilized lactoferrin comprises a combination of branched chain amino acids and beta-lactoferrin.

6. The composition according to claim 1, said composition further comprising a combination of branched chain amino acids.

7. The composition according to claim 6, said wherein said branched chain amino acids are L-leucine, L-isoleucine and L-valine.

8. The composition according to claim 7, wherein said amino acids are present at a ratio of 2:1:1 (L-leucine:L-isoleucine:L-valine).

9. The composition according to claim 2, said composition further comprising a combination of branched chain amino acids.

10. The composition according to claim 9, said wherein said branched chain amino acids are L-leucine, L-isoleucine and L-valine.

11. The composition according to claim 10, wherein said amino acids are present at a ratio of 2:1:1 (L-leucine:L-isoleucine:L-valine).

12. The composition according to claim 1, said composition further comprising a nonionic surface-active emulsifier selected from the group consisting of:
a) mono and diglycerides of the fatty acid oleic acid;
b) polyglyceride esters of the fatty acid oleic acid;
c) mono, di, and polyglyceride esters of the fatty acid oleic acid further esterified with a dibasic organic acid selected from the group consisting of citric and lactic acids;
d) acetylated mono, di, and polyglyceride esters of the fatty acid oleic acid further esterified with a dibasic organic acid selected from the group consisting of citric and lactic acids;
e) sorbitan esters of the fatty acid oleic acid; and
f) any combination of a-c.

13. The composition according to claim 1, said composition further comprising a natural antioxidant selected from the group consisting of:
a) Rosemary extracts;
b) tocopherols;
c) ascorbic acid;
d) ascorbyl palmitate;
e) phospholipids; and
f) combinations thereof.

14. The composition according to claim 1, said composition further comprising a heat-stable sweetener selected from the group consisting of:
a) sucralose;
b) acesulfame potassium; and
c) sugar alcohols.

15. The composition according to claim 1, said composition further comprising an edible oil blend comprising ω-3, ω-6 and ω-9 fatty acids.

16. The composition according to claim 1, said composition comprising one or more anti-aging agent selected from the group consisting of:
a) coenzyme Q₁₀;
b) a xanthophyll, such as astaxanthin, fucoxanthin and/or zeaxanthin;
c) L-Glutathione; and
d) leutein.

17. The composition according to claim 1, said composition further comprising a polysaccharide selected from the group consisting of:
a) alginates;
b) carrageenans;
c) dextrans;
d) galactomannans;
e) glucomanans;
f) kefiran;
g) pectin;
h) starch; and
i) combinations thereof.

18. The composition according to claim 16, wherein said anti-aging agent is encapsulated with a polysaccharide selected from the group consisting of:
a) alginates;
b) carrageenans;
c) dextrans;
d) galactomannans;
e) glucomanans;
f) kefiran;
g) pectin;
h) starch; and
i) combinations thereof.

19. The composition according to claim 1, said composition comprising:
(a) heat stabilized lactoferrin, heat stabilized lactoglobulin or a combination of heat stabilized lactoferrin and heat stabilized lactoglobulin;
(b) a buffer selected from the group consisting of acetate salts of:
i) sodium,
ii) potassium,
iii) magnesium, and
iv) calcium;
(c) one or more anti-aging agent selected from the group consisting of:
i) coenzyme Q₁₀;
ii) a xanthophyll, such as astaxanthin, fucoxanthin and/or zeaxanthin; and
iii) L-Glutathione;
iv) leutein; and
(d) a nonionic surface-active emulsifier selected from the group consisting of:
i) mono and diglycerides of the fatty acid oleic acid;
ii) polyglyceride esters of the fatty acid oleic acid;
iii) mono, di, and polyglyceride esters of the fatty acid oleic acid further esterified with a dibasic organic acid selected from the group consisting of citric and lactic acids;
iv) acetylated mono, di, and polyglyceride esters of the fatty acid oleic acid further esterified with a dibasic organic acid selected from the group consisting of citric and lactic acids;
v) sorbitan esters of the fatty acid oleic acid; and
vi) any combination of i-v.

20. The composition according to claim 19, said composition further comprising:
(a) a natural antioxidant selected from the group consisting of:
i) Rosemary extracts;
ii) tocopherols;
iii) ascorbic acid;
iv) ascorbyl palmitate;
v) phospholipids; and
vi) combinations thereof;
(b) an edible oil blend comprising ω-3, ω-6 and ω-9 fatty acids; and
(c) a polysaccharide selected from the group consisting of:
i) alginates;
ii) carrageenans;
iii) dextrans;
iv) galactomannans;
v) glucomanans;
vi) kefiran;
vii) pectin;
viii) starch; and
ix) combinations thereof.

21. The composition according to claim 19, said composition further comprising a heat-stable sweetener selected from the group consisting of:
a) sucralose;
b) acesulfame potassium; and
c) sugar alcohols.

22. The composition according to claim 19, said composition further comprising a combination of branched chain amino acids.

23. The composition according to claim 22, said wherein said branched chain amino acids are L-leucine, L-isoleucine and L-valine.

24. The composition according to claim 23, wherein said amino acids are present at a ratio of 2:1:1 (L-leucine:L-isoleucine:L-valine).

25. A method of preparing heat stabilized lactoferrin comprising heating a mixture of lactoferrin, zinc lactate and, optionally, branched-chain amino acids to a temperature between about 90° C. and about 150° C. for a period of about 30 seconds to less than 5 minutes, about 30 seconds to less than sixty minutes or a period of about 30 seconds to one day or more.

26. The method according to claim 25, wherein said mixture contains branched chain amino acids, said branched chain amino acids comprising L-leucine, L-isoleucine and L-valine.

27. The method according to claim 26, wherein said amino acids are present at a ratio of 2:1:1 (L-leucine:L-isoleucine:L-valine).

28. A method for stabilizing an anti-aging agent comprising emulsifying one or more anti-aging agent to a composition comprising:
(a) heat stabilized lactoferrin, heat stabilized lactoglobulin or a combination of heat stabilized lactoferrin and heat stabilized lactoglobulin;
(b) a buffer selected from the group consisting of acetate salts of:
i) sodium,
ii) potassium,
iii) magnesium, and
iv) calcium; and
(c) a nonionic surface-active emulsifier selected from the group consisting of:
i) mono and diglycerides of the fatty acid oleic acid;
ii) polyglyceride esters of the fatty acid oleic acid;
iii) mono, di, and polyglyceride esters of the fatty acid oleic acid further esterified with a dibasic organic acid selected from the group consisting of citric and lactic acids;
iv) acetylated mono, di, and polyglyceride esters of the fatty acid oleic acid further esterified with a dibasic organic acid selected from the group consisting of citric and lactic acids;
v) sorbitan esters of the fatty acid oleic acid; and
vi) any combination of i-v.

29. The method according to claim 28, wherein said one or more anti-aging agent is selected from the group consisting of:
a) coenzyme Q₁₀;
b) a xanthophyll, such as astaxanthin, fucoxanthin and/or zeaxanthin;
c) L-Glutathione; and
d) leutein.

30. The method according to claim 28, wherein said anti-aging agent is encapsulated in a polysaccharide selected from the group consisting of:
i) alginates;
ii) carrageenans;
iii) dextrans;
iv) galactomannans;
v) glucomanans;
vi) kefiran;
vii) pectin;
viii) starch; and
ix) combinations thereof.

31. A method for delivering an anti-aging agent to a human or animal, said method comprising administering a therapeutically effective amount of a composition according to claim 1 to a human or animal.

32. A method of immunizing a human or animal comprising administering a composition comprising heat stabilized lactoferrin and a vaccine to human or animal.

33. The method according to claim 32, wherein the production of IL-2 is increased in said human or animal.

34. A composition exhibiting antioxidant activity for protection against oxidative damage at the cellular level comprising:
(a) branched-chain amino acids and whey protein; and
(b) one or more anti-aging agent selected from the group consisting of:
i) coenzyme Q₁₀;
ii) xanthophylls such as lutein, astaxanthin, fucoxanthin and/or zeaxanthin; and
iii) L-Glutathione.

35. The composition according to claim 34, wherein said composition comprises branched-chain amino acids.

36. The composition according to claim 35, wherein said branched-chain amino acids are L-Leucine, L-Isoleucine and L-Valine.

37. The composition according to claim 35, wherein said L-Leucine, L-Isoleucine and L-Valine are present at a ratio of 2:1:1.

38. The composition according to claim 34, wherein said the whey protein is lactoferrin.

39. The composition according to claim 38, wherein said the lactoferrin is heat-stabilized.

40. The composition according to claim 34, wherein said the whey protein is β-lactoglobulin.

41. The composition according to claim 34, said composition further comprising a buffer selected from the group consisting of acetate salts of: p1 a) sodium,
b) potassium,
c) magnesium,
d) calcium; and
e) any combination of a-d.

42. The composition according to claim 34, said composition further comprising a nonionic surface-active emulsifier selected from the group consisting of:
a) mono and diglycerides of the fatty acid oleic acid,
b) polyglyceride esters of the fatty acid oleic acid,
c) mono, di, and polyglyceride esters of the fatty acid oleic acid further esterified with a dibasic organic acid selected from the group consisting of citric and lactic acids,
d) acetylated mono, di, and polyglyceride esters of the fatty acid oleic acid further esterified with a dibasic organic acid selected from the group consisting of citric and lactic acids,
e) sorbitan esters of the fatty acid oleic acid; and
f) any combination of a-e.

43. The composition according to claim 34, said composition further comprising a natural antioxidant selected from the group consisting of:
a) Rosemary extracts,
b) tocopherols,
c) ascorbic acid,
d) ascorbyl palmitate,
e) phospholipids; and
f) any combination of a-e.

44. The composition according to claim 34, said composition further comprising a heat-stable sweetener selected from the group consisting of:
a) sucralose,
b) acesulfame potassium,
c) sugar alcohols; and
d) any combination of a-c.

45. The composition according to claim 34, said composition further comprising an edible oil blend consisting of ω-3, ω-6 and ω-9 fatty acids.

46. The composition according to claim 34, said composition further comprising a polysaccharide selected from the group consisting of:
a) alginates,
b) carrageenans,
c) dextrans,
d) galactomanans,
e) glucomanans,
f) kefiran,
g) pectin,
h) starch; and
i) any combination of a-h.

47. The composition of claim 34, wherein said heat-stabilized lactoferrin comprises a mixture of lactoferrin, natural zinc lactate, and optionally, branched-chain amino acids.

48. A method to stabilize lactoferrin comprising combining lactoferrin zinc lactate and branched-chain amino acids at a substantially neutral pH and heating the composition to about 90° C.

49. A method to form stable β-lactoglobulin microcarriers comprising combining β-lactoglobulin and L-arginine and heating the composition to a temperature of about 90° C.

50. A method for delaying aging in humans or animals, said method comprising administering a therapeutically effective amount of the anti-aging agents of claim 34, wherein said the anti-aging agents are capable of delaying aging in humans or animals.

51. A method to augment vaccine efficacy against infections comprising administering a composition comprising heat-stabilized lactoferrin according to claim 34 and a vaccine to a subject (human or animal) that is to be immunized.

52. The method according to claim 51, wherein said heat-stabilized lactoferrin comprises a mixture of lactoferrin, natural zinc lactate, and optionally, branched-chain amino acids

53. A method for delaying aging in humans or animals, said method comprising administering a therapeutically effective amount of the composition of claim 34, wherein said composition delays aging in humans or animals by augmenting vaccine efficacy against infections.
